SEQUENCE LISTING (1 sequence)

SEQ ID NO: 1 - protein, 491 aa, Homo sapiens
MGCIKSKGKDSLSDDGVDLKTQPVPESQLLPGQRFQTKDPEEQGDIVVALYPYDGIHPDDLSFKKGEKMKVLEEHGEWWKAKSLLTKKEGFIPSNYVAKLNTLETEEWFFKDITRKDAERQLLAPGNSAGAFLIRESETLKGSFSLSVRDFDPVHGDVIKHYKIRSLDNGGYYISPRITFPCISDMIKHYQKQADGLCRRLEKACISPKPQKPWDKDAWEIPRESIKLVKRLGAGQFGEVWMGYYNNSTKVAVKTLKPGTMSVQAFLEEANLMKTLQHDKLVRLYAVVTREEPIYIITEYMAKGSLLDFLKSDEGGKVLLPKLIDFSAQIAEGMAYIERKNYIHRDLRAANVLVSESLMCKIADFGLARVIEDNEYTAREGAKFPIKWTAPEAINFGCFTIKSDVWSFGILLYEIVTYGKIPYPGRTNADVMTALSQGYRMPRVENCPDELYDIMKMCWKEKAEERPTFDYLQSVLDDFYTATEGQYQQQP

(19) United States
(12) Patent Application Publication
(10) Pub. No.: US 2013/0203789 A1
(43) Pub. Date: Aug. 8, 2013

(54) Novel Inhibitors of LYN Kinase and Methods Using Same

(71) Applicant: VASSA INFORMATICS, Texarkana, AR (US)

(72) Inventors: Gerald Wyckoff, Overland Park, KS (US); Brian J. Moldover, Philadelphia, PA (US); Lisa Kenney, El Cerrito, CA (US); Ada Solidar, Kanasa City, MO (US)

(73) Assignee: VASSA INFORMATICS, Texarkana, AR (US)

(21) Appl. No.: 13/763,086

(22) Filed: Feb. 8, 2013

Related U.S. Application Data
(60) Provisional application No. 61/596,416, filed on Feb. 8, 2012.

(51) Int. Cl. C07D 471/14

(52) U.S. Cl. CPC C07D 471/14
USPC 514/267; 544/251

(57) ABSTRACT

The invention includes compounds that inhibit LYN kinase activity. The invention further includes a method of treating, ameliorating or preventing cancer in a subject in need thereof, wherein the cancer is dependent on LYN kinase activity.

### CROSS-REFERENCE TO RELATED APPLICATION

The present application claims the benefit of U.S. Provisional Patent Application No. 61/596,416, filed Feb. 8, 2012, which application is hereby incorporated by reference in its entirety herein.

### BACKGROUND OF THE INVENTION

LYN kinase (protein sequence SEQ ID NO:1) is a member of the Src family of protein tyrosine kinases, and is mainly expressed in hematopoietic cells and in neural tissues (Yamanashi et al., 1989, Proc. Natl. Acad. Sci. U.S.A. 86(17):6538-42; Umemori et al., 1992, Brain Res. Mol. Brain. Res. 16(3-4): 303-10).

In various hematopoietic cells, LYN kinase has emerged as a key enzyme involved in the regulation of cell activation. In these cells, a small amount of LYN kinase is associated with cell surface receptor proteins, including the B cell antigen receptor (BCR), CD40, or CD19 (Yamamoto et al., 1993, Immunol. Rev. 132:187-206; Campbell & Sefton, 1992, Cell. Biol. 12 (5):2315-21; Ren et al., 1994, J. Exp. Med. 179 (2): 673-80).

Following engagement of the B cell receptors, LYN kinase undergoes rapid phosphorylation and activation. LYN kinase activation triggers a cascade of signaling events mediated by LYN kinase phosphorylation of tyrosine residues within the immunoreceptor tyrosine-based activation motifs (ITAM) of the receptor proteins, and subsequent recruitment and activation of other kinases including Syk, phospholipase Cγ2 (PLCγ2) and phosphatidyl inositol-3 kinase (Yamanishi et al., 1992, Proc. Natl. Acad. Sci. 89(3):1118-22). These kinases provide activation signals, which play critical roles in proliferation, Ca²⁺ mobilization and cell differentiation.

LYN kinase also plays an essential role in the transmission of inhibitory signals through phosphorylation of tyrosine residues within the immunoreceptor tyrosine-based inhibitory motifs (ITIM) in regulatory proteins such as CD22, PIR-B and FCγRIIb1. Their ITIM phosphorylation subsequently leads to recruitment and activation of phosphatases such as SHIP-1 and SHP-1 (Cornall et al., 1998, Immunity 8(4):497-508; Smith et al., 1998, J. Exp. Med. 187(5):807-11; Chan et al., 1998, Curr. Biol. 8(10):545-53; Nishizumi et al., 1998, J. Exp. Med. 187(8):1343-48; Maeda et al., 1999, Oncogene 18(14):2291-97), which further down modulate signaling pathways, attenuate cell activation and can mediate tolerance.

By acting as a key regulator of both positive and negative signals, LYN kinase plays an important role in B cells, setting the threshold of cell signaling and maintaining the balance between activation and inhibition. LYN kinase thus functions as a rheostat that modulates signaling rather than as a binary on-off switch (Lowell, 2004, Mol. Immunol. 41(6-7):631-43; Saijo et al., 2003, Nat. Immunol. 4(3):274-79; Xu et al., 2005, Immunity 22 (1):9-18).

LYN kinase deficient mice display a phenotype that includes reduced numbers of mature B cells, B cell hyper-responsiveness to BCR stimulation, elevated serum IgM levels, accumulation of autoimmune antibodies, and development of autoimmune glomerulonephritis in aging mice. Mice expressing a hyperactive LYN kinase allele also develop severe autoimmune glomerulonephritis and have a reduced life expectancy. Deregulation of the balance between positive vs. negative signaling is thought to underlie the paradoxical observation of B cell hyperactivity and lethal autoimmune glomerulonephritis in mice with reduced or elevated LYN kinase activity.

LYN kinase activation has been correlated to resistance to imatinib (also known as Gleevec™ or 4-[(4-methylpiperazin-1-yl)methyl]-N-[4-methyl-3-[(4-pyridin-3-ylpyrimidin-2-yl)amino]phenyl]benzamide). Imatinib is a tyrosine kinase inhibitor used to treat chronic myelogenous leukemia (CML). BCR-ABL mutations are associated with failure of imatinib treatment in many CML patients. LYN kinase regulates survival and responsiveness of CML cells to inhibition of BCR-ABL kinase, and differences in LYN kinase regulation have been found between imatinib-sensitive and imatinib-resistant CML cell lines. Activation of LYN kinase appears to play a direct role in the survival of CML cells that are resistant to imatinib mesylate (J. Natl. Cancer Inst, 2008, 100(13):926-39).

LYN kinase has also been implicated in breast cancer, where it is likely a target for dasatinib, a dual-specificity tyrosine kinase inhibitor (Sprycel™; N-(2-chloro-6-methylphenyl)-2-[[6-[4-(2-hydroxyethyl)-1-piperazinyl]-2-methyl-4-pyrimidinyl]amino]-5-thiazole carboxamide). In mesenchymal breast cancer lines, RNAi-mediated knockdown of LYN kinase inhibited cell migration and invasion, but not proliferation. Dasatinib also blocked invasion (but not proliferation) at concentrations that inhibit LYN kinase activity, suggesting that LYN kinase plays an important role in invasion and that invasion may be used as a relevant end point for dasatinib therapy. Use of LYN kinase as a therapeutic agent has particular relevance to treatment of clinically aggressive basal-like breast cancer (Cancer Res., 70(6):2296-306). In clinical outcome measures for breast cancer patients, LYN kinase activation was associated with significantly shorter overall survival (P=0.02).

LYN kinase is also the primary SRC kinase expressed in glioblastoma. Glioblastoma is the most common and most aggressive type of primary brain tumor in humans, involving glial cells and accounting for 52% of all parenchymal brain tumor cases and 20% of all intracranial tumors. Glioblastoma has the worst prognosis of any CNS malignancy, with median survival of about 14 months. LYN kinase activity is significantly elevated in glioblastoma (grade IV) tumor biopsies compared with anaplastic astrocytoma (grade III) tumor biopsies, non-neoplastic brain biopsies, and normal autopsy brain cortex and white matter. The strong expression of LYN protein in the glioblastoma cells indicates that LYN kinase activity is poised to promote the highly migratory/invasive phenotype of glioblastoma tumors (Cancer Res., 2005, 65:5535).

Inhibition of LYN kinase activity also contributes to inhibition of tumor growth and metastasis in Ewing's sarcoma. Ewing's sarcoma is a poorly differentiated pediatric and young adult cancer of bone and soft tissue, accounting for 30% of bone cancers in children. A treatment program for Ewing's sarcoma may include several approaches such as surgery, radiation and chemotherapy. High c-Src kinase activity has been reported in Ewing's sarcoma cell lines compared with normal human fibroblasts and other tumor cell lines. Further analysis indicated that LYN kinase was the primary Src expressed in those cell lines. Down-regulation of LYN kinase activity by siRNA or by treatment with the inhibitor AP23994 (Ariad) inhibited tumor growth and the metastatic behavior in vitro and in vivo. Inhibitors of LYN kinase may thus represent a potentially new treatment modality for Ewing's sarcoma (Mol. Cancer. Ther., 2008, 7(7):1807-16).

Interestingly, no LYN-specific inhibitors have been disclosed or marketed so far. Dasatinib (Sprycel™, BMS, NYC, NY) is a multi BCR-ABL/Src kinase inhibitor and is only approved for use for use in patients with CML who have failed treatment with imatinib. Ponatinib (Ariad, Cambridge, Mass.) is a multi BCR-ABL/Src kinase inhibitor in phase II trials for treatment of chronic myelogenic leukemia. Bafetinib (CytRx, Los Angeles, Calif.) is a potent and specific inhibitor of BCR-ABL and LYN kinases, now being tested in patients with high-risk B-cell chronic lymphocytic leukemia (B-CLL) and advanced prostate cancer.

There remains a need in the art to identify novel potent and selective inhibitors of LYN kinase. Such inhibitors may be used in the treatment of cancers such as CML, B-CLL, glioblastoma, breast cancer, Ewing's sarcoma and prostate cancer. The present invention fills this need.

### BRIEF SUMMARY OF THE INVENTION

The invention includes a compound of formula (I):

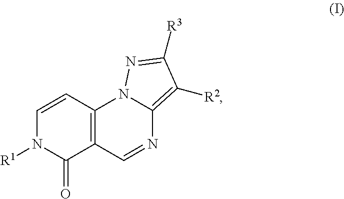

or a salt thereof, wherein: R¹ and R⁶ are independently unsubstituted or substituted alkyl, unsubstituted or substituted aryl, or unsubstituted or substituted heteroaryl; and R³ is H or C₁-C₆ alkyl; with the proviso that the compound of formula (I) is not 7-(2-fluorophenyl)-3-(4-fluorophenyl)-2-methylpyrazolo[1,5-a]pyrido[3,4-e]pyrimidin-6(7H)-one; 3-(4-chlorophenyl)-7-(2-fluorophenyl)-2-methylpyrazolo[1,5-a]pyrido[3,4-e]pyrimidin-6(7H)-one; 7-(2-fluorophenyl)-2-methyl-3-phenylpyrazolo[1,5-a]pyrido[3,4-e]pyrimidin-6(7H)-one; 7-(3-fluorophenyl)-2-methyl-3-phenylpyrazolo[1,5-a]pyrido[3,4-e]pyrimidin-6(7H)-one; 7-(3-chloro-4-fluorophenyl)-2-methyl-3-phenylpyrazolo[1,5-a]pyrido[3,4-e]pyrimidin-6(7H)-one; 7-(3,4-difluorophenyl)-2-methyl-3-phenylpyrazolo[1,5-a]pyrido[3,4-e]pyrimidin-6(7H)-one; 7-(3-chlorophenyl)-3-(4-fluorophenyl)-2-methylpyrazolo[1,5-a]pyrido[3,4-e]pyrimidin-6(7H)-one; 7-(2-chlorophenyl)-3-(4-fluorophenyl)-2-methylpyrazolo[1,5-a]pyrido[3,4-e]pyrimidin-6(7H)-one; 3-(4-fluorophenyl)-7-(2-methoxyphenyl)-2-methylpyrazolo[1,5-a]pyrido[3,4-e]pyrimidin-6(7H)-one; 2-(3-(4-fluorophenyl)-2-methyl-6-oxopyrazolo[1,5-a]pyrido[3,4-e]pyrimidin-7(6H)-yl)benzonitrile; 2-ethyl-3-(4-fluorophenyl)-7-phenylpyrazolo[1,5-a]pyrido[3,4-e]pyrimidin-6(7H)-one; or a salt thereof.

In one embodiment, R¹ and R² are independently unsubstituted or substituted aryl, or unsubstituted or substituted heteroaryl. In another embodiment, R³ is H, methyl or ethyl.

The invention further includes a method of inhibiting LYN kinase activity in a subject in need thereof. The method comprises administering to the subject a pharmaceutical composition comprising a pharmaceutically acceptable carrier and a therapeutically effective amount of a compound of formula (II):

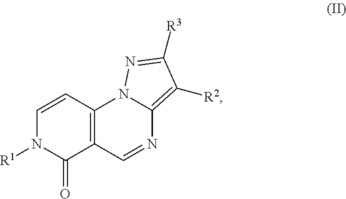

or a salt thereof, wherein: R¹ and R⁶ are independently unsubstituted or substituted alkyl, unsubstituted or substituted aryl, or unsubstituted or substituted heteroaryl; and R³ is H or C₁-C₆ alkyl; whereby the administration of the composition inhibits LYN kinase activity in the subject.

In one embodiment, the compound of formula (II) is selected from the group consisting of:
7-(2-fluorophenyl)-3-(4-fluorophenyl)-2-methylpyrazolo[1,5-a]pyrido[3,4-e]pyrimidin-6(7H)-one:

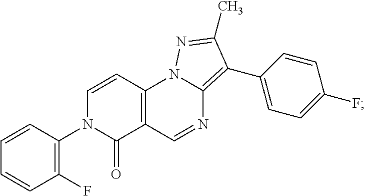 [SMILES: Cc1nn2c(ncc3c(=O)n(-c4ccccc4F)ccc32)c1-c1ccc(F)cc1]

3-(4-chlorophenyl)-7-(2-fluorophenyl)-2-methylpyrazolo[1,5-a]pyrido[3,4-e]pyrimidin-6(7H)-one:

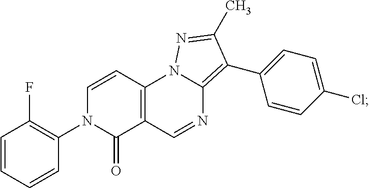 [SMILES: Cc1nn2c(ncc3c(=O)n(-c4ccccc4F)ccc32)c1-c1ccc(Cl)cc1]

7-(2-fluorophenyl)-2-methyl-3-phenylpyrazolo[1,5-a]pyrido[3,4-e]pyrimidin-6(7H)-one:

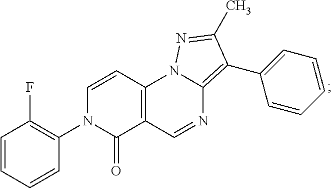 [SMILES: Cc1nn2c(ncc3c(=O)n(-c4ccccc4F)ccc32)c1-c1ccccc1]

7-(3-fluorophenyl)-2-methyl-3-phenylpyrazolo[1,5-a]pyrido[3,4-e]pyrimidin-6(7H)-one:

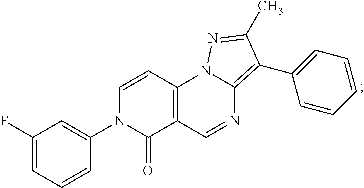 [SMILES: Cc1nn2c(ncc3c(=O)n(-c4cccc(F)c4)ccc32)c1-c1ccccc1]

7-(3-chloro-4-fluorophenyl)-2-methyl-3-phenylpyrazolo[1,5-a]pyrido[3,4-e]pyrimidin-6(7H)-one:

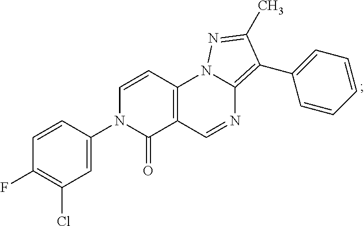 [SMILES: Cc1nn2c(ncc3c(=O)n(-c4ccc(F)c(Cl)c4)ccc32)c1-c1ccccc1]

7-(3,4-difluorophenyl)-2-methyl-3-phenylpyrazolo[1,5-a]pyrido[3,4-e]pyrimidin-6(7H)-one:

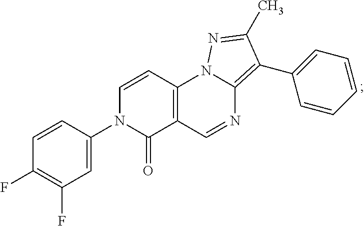 [SMILES: Cc1nn2c(ncc3c(=O)n(-c4ccc(F)c(F)c4)ccc32)c1-c1ccccc1]

7-(3-chlorophenyl)-3-(4-fluorophenyl)-2-methylpyrazolo[1,5-a]pyrido[3,4-e]pyrimidin-6(7H)-one:

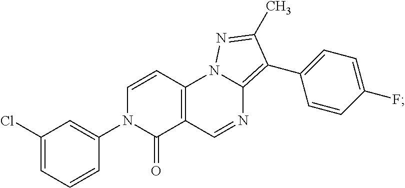 [SMILES: Cc1nn2c(ncc3c(=O)n(-c4cccc(Cl)c4)ccc32)c1-c1ccc(F)cc1]

7-(2-chlorophenyl)-3-(4-fluorophenyl)-2-methylpyrazolo[1,5-a]pyrido[3,4-e]pyrimidin-6(7H)-one:

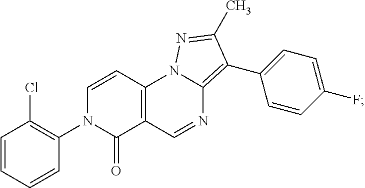 [SMILES: Cc1nn2c(ncc3c(=O)n(-c4ccccc4Cl)ccc32)c1-c1ccc(F)cc1]

3-(4-fluorophenyl)-7-(2-methoxyphenyl)-2-methylpyrazolo[1,5-a]pyrido[3,4-e]pyrimidin-6(7H)-one:

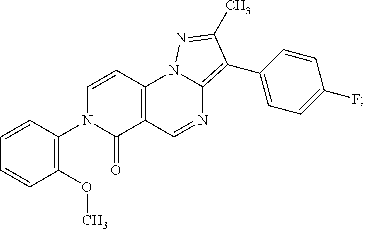 [SMILES: COc1ccccc1-n1ccc2c(cnc3c(-c4ccc(F)cc4)c(C)nn32)c1=O]

2-(3-(4-fluorophenyl)-2-methyl-6-oxopyrazolo[1,5-a]pyrido[3,4-e]pyrimidin-7(6H)-yl)benzonitrile:

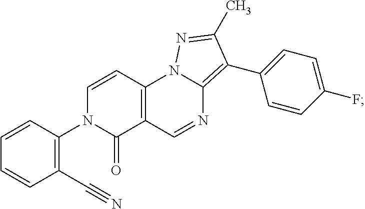 [SMILES: Cc1nn2c(ncc3c(=O)n(-c4ccccc4C#N)ccc32)c1-c1ccc(F)cc1]

2-ethyl-3-(4-fluorophenyl)-7-phenylpyrazolo[1,5-a]pyrido[3,4-e]pyrimidin-6(7H)-one:

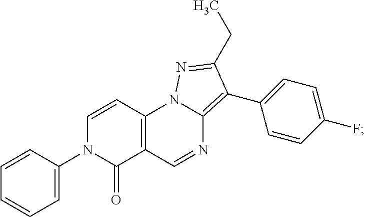 [SMILES: CCc1nn2c(ncc3c(=O)n(-c4ccccc4)ccc32)c1-c1ccc(F)cc1]

and a salt thereof. In another embodiment, the subject is human.

The invention further includes a method of treating, ameliorating or preventing cancer in a subject in need thereof, the method comprising administering to the subject a pharmaceutical composition comprising a pharmaceutically acceptable carrier and a therapeutically effective amount of a compound of formula (II):

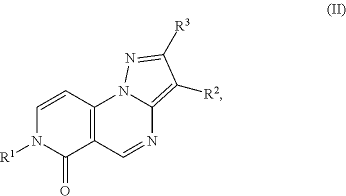

or a salt thereof, wherein: R¹ and R⁶ are independently unsubstituted or substituted alkyl, unsubstituted or substituted aryl, or unsubstituted or substituted heteroaryl; and R³ is H or C₁-C₆ alkyl; wherein the cancer is dependent on LYN kinase activity, whereby administration of the composition inhibits the LYN kinase activity and treats, ameliorates or prevent the cancer in the subject.

In one embodiment, the compound of formula (II) is selected from the group consisting of:
7-(2-fluorophenyl)-3-(4-fluorophenyl)-2-methylpyrazolo[1,5-a]pyrido[3,4-e]pyrimidin-6(7H)-one:

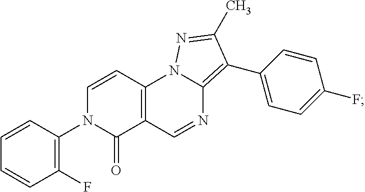 [SMILES: Cc1nn2c(ncc3c(=O)n(-c4ccccc4F)ccc32)c1-c1ccc(F)cc1]

3-(4-chlorophenyl)-7-(2-fluorophenyl)-2-methylpyrazolo[1,5-a]pyrido[3,4-e]pyrimidin-6(7H)-one:

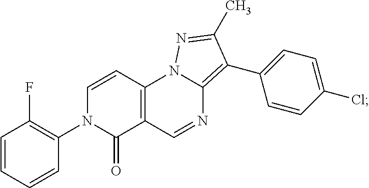 [SMILES: Cc1nn2c(ncc3c(=O)n(-c4ccccc4F)ccc32)c1-c1ccc(Cl)cc1]

7-(2-fluorophenyl)-2-methyl-3-phenylpyrazolo[1,5-a]pyrido[3,4-e]pyrimidin-6(7H)-one:

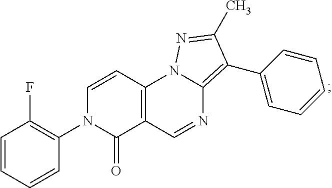 [SMILES: Cc1nn2c(ncc3c(=O)n(-c4ccccc4F)ccc32)c1-c1ccccc1]

7-(3-fluorophenyl)-2-methyl-3-phenylpyrazolo[1,5-a]pyrido[3,4-e]pyrimidin-6(7H)-one:

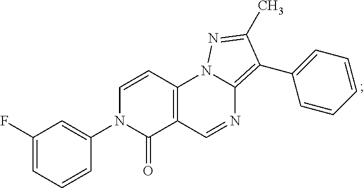 [SMILES: Cc1nn2c(ncc3c(=O)n(-c4cccc(F)c4)ccc32)c1-c1ccccc1]

7-(3-chloro-4-fluorophenyl)-2-methyl-3-phenylpyrazolo[1,5-a]pyrido[3,4-e]pyrimidin-6(7H)-one:

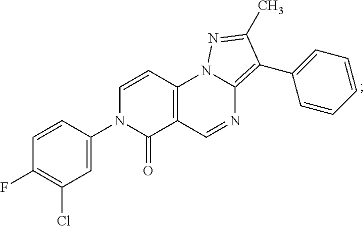 [SMILES: Cc1nn2c(ncc3c(=O)n(-c4ccc(F)c(Cl)c4)ccc32)c1-c1ccccc1]

7-(3,4-difluorophenyl)-2-methyl-3-phenylpyrazolo[1,5-a]pyrido[3,4-e]pyrimidin-6(7H)-one:

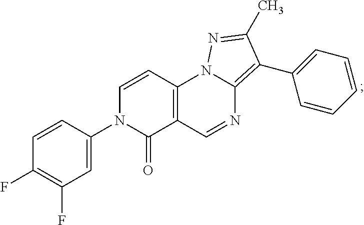 [SMILES: Cc1nn2c(ncc3c(=O)n(-c4ccc(F)c(F)c4)ccc32)c1-c1ccccc1]

7-(3-chlorophenyl)-3-(4-fluorophenyl)-2-methylpyrazolo[1,5-a]pyrido[3,4-e]pyrimidin-6(7H)-one:

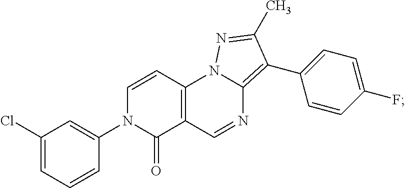 [SMILES: Cc1nn2c(ncc3c(=O)n(-c4cccc(Cl)c4)ccc32)c1-c1ccc(F)cc1]

7-(2-chlorophenyl)-3-(4-fluorophenyl)-2-methylpyrazolo[1,5-a]pyrido[3,4-e]pyrimidin-6(7H)-one:

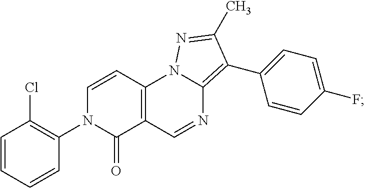 [SMILES: Cc1nn2c(ncc3c(=O)n(-c4ccccc4Cl)ccc32)c1-c1ccc(F)cc1]

3-(4-fluorophenyl)-7-(2-methoxyphenyl)-2-methylpyrazolo[1,5-a]pyrido[3,4-e]pyrimidin-6(7H)-one:

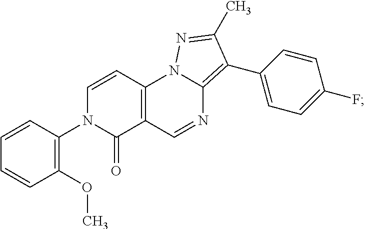 [SMILES: COc1ccccc1-n1ccc2c(cnc3c(-c4ccc(F)cc4)c(C)nn32)c1=O]

2-(3-(4-fluorophenyl)-2-methyl-6-oxopyrazolo[1,5-a]pyrido[3,4-e]pyrimidin-7(6H)-yl)benzonitrile:

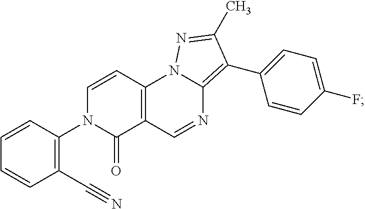 [SMILES: Cc1nn2c(ncc3c(=O)n(-c4ccccc4C#N)ccc32)c1-c1ccc(F)cc1]

2-ethyl-3-(4-fluorophenyl)-7-phenylpyrazolo[1,5-a]pyrido[3,4-e]pyrimidin-6(7H)-one:

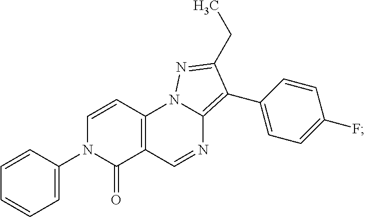 [SMILES: CCc1nn2c(ncc3c(=O)n(-c4ccccc4)ccc32)c1-c1ccc(F)cc1]

and a salt thereof.

In one embodiment, the subject is human. In another embodiment, the cancer is selected from the group consisting of CML, B-CLL, glioblastoma, breast cancer, Ewing's sarcoma and prostate cancer. In yet another embodiment, the CML cancer is resistant to 4-[(4-methylpiperazin-1-yl)methyl]-N-[4-methyl-3-[(4-pyridin-3-ylpyrimidin-2-yl)amino]phenyl]benzamide (imatinib).

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIG. 1 is a graph illustrating the imatinib and dasatinib treatment and survival of K562 (squares), LYN-overexpressing (pMX-LYN-transfected) K562 (triangles), K562R (inverted triangles), and control K562 cells transfected with empty vector (pMX) (diamonds).

FIG. 2 is a drawing illustrating LYN regulation in “normal” and LYN-overexpressing CML cells. LYN expression and activation are heterogeneous in CML cells and may be altered by imatinib therapy. In early stage or untreated (“normal”) CML, BCR-ABL is upstream of LYN and other Src-family kinases. BCR-ABL-mediated LYN (or related kinase) activation provides essential, possibly lineage-specific, support for BCR-ABL-mediated transformation. In this setting, BCR-ABL inhibition (with imatinib) reduces LYN kinase activation and loss of signaling through BCR-ABL- and LYN-mediated phosphorylation. Imatinib exposure alters LYN expression or induces changes in upstream control of its activation. LYN activation acquires full or partial BCR-ABL independence, resulting in loss of control of LYN activation with imatinib alone. Unregulated LYN alters BCR-ABL signaling complexes, associating with Gab2, inducing its tyrosine phosphorylation, and maintaining or directing Y177 phosphorylation of BCR-ABL. These changes suppress or delay kinase inhibition by imatinib (Blood, 2008, 111(7):3821-29).

FIG. 3, comprising FIGS. 3A-3B, illustrates the relationship between LYN expression and unfavorable outcome in cancers. FIG. 3A illustrates LYN immunostaining on TMA. Representative breast cancer cases negative (arrow, region with tumor cells) or positive for expression of the LYN tyrosine kinase in tumor cells. FIG. 3B illustrates that LYN expression in tumor cells predicts unfavorable outcome. Kaplan-Meier analysis comparing LYN-positive (LYN+) and LYN-negative (LYN−) cases for relapse-free survival (top) and overall survival (bottom); P values are shown.

FIG. 4, comprising FIGS. 4A-4C, illustrates the finding that dasatinib inhibits cell invasion at concentrations that inhibit LYN. Dasatinib effect (dose-response curve) on (FIG. 4A) cell viability and (FIG. 4B) cell invasion for BT549 cells (left) and Hs578T cells (right). IC₅₀ values (indicated) were determined from sigmoidal (four-parameter logistic) curves. FIG. 4C: verification in BT549 cells that dasatinib treatment leads to decreased phosphorylated (activated) LYN, assayed by Western blot.

FIG. 5, comprising FIGS. 5A-5B, is a set of graphs illustrating elevated levels of LYN activity in glioblastoma tumor samples. FIG. 5A: columns, mean for % LYN activity and % non-LYN activity for each sample group; bars, SD. FIG. 5B: normalized level of LYN activity. Horizontal bars, median levels of normalized LYN activity: 21.4 for non-neoplastic brain, 40.5 for normal autopsy brain, 52.1 for grade III tumors, and 124.1 for glioblastoma tumors.

FIG. 6 is a set of figures illustrating that LYN protein is strongly expressed in glioblastoma tumor cells in biopsy samples. Black arrows, endothelial cells; white arrowheads, glioblastoma tumor cells. Magnification, ×250 (A-E).

FIG. 7, comprising FIGS. 7A-7B, illustrates the effect of the SFK inhibitor AP23994 on LYN kinase activity and cell proliferation. As illustrated in FIG. 7A, TC71 cells were treated with 0, 0.5, 1, or 2 nmol/L AP23994 for 1 h. Cell lysates were collected and assayed for LYN kinase. As illustrated in FIG. 7B, TC71 cells were treated with various doses of AP23994 or DMSO for 48 h and then assayed by MTT.

FIG. 8 is a scheme illustrating the role that LYN kinase plays in B-Cell signaling and immune response.

FIG. 9 is a schematic kinome tree illustrating the evolutionary relationships between 299 kinases. The inhibition of 68 representative kinases by VI301 at 100 μM concentration was evaluated. VI301 was found to inhibit LYN kinase by 95%, CamK1 by 26%, Pim-1 kinase by 12%, and WNK3 kinase by 3%. Very high (106%) inhibition was seen to Abl kinase (BCR-Abl).

FIG. 10, comprising FIGS. 10A-10B, illustrates the proposed binding model of VI301 to LYN kinase. FIG. 10A is a representation of the structure of VI301, and FIG. 10B is a representation of the proposed model for binding of VI301 to LYN kinase. The affinity of KLS2 for LYN kinase was calculated to be 10.1 kcal/mol.

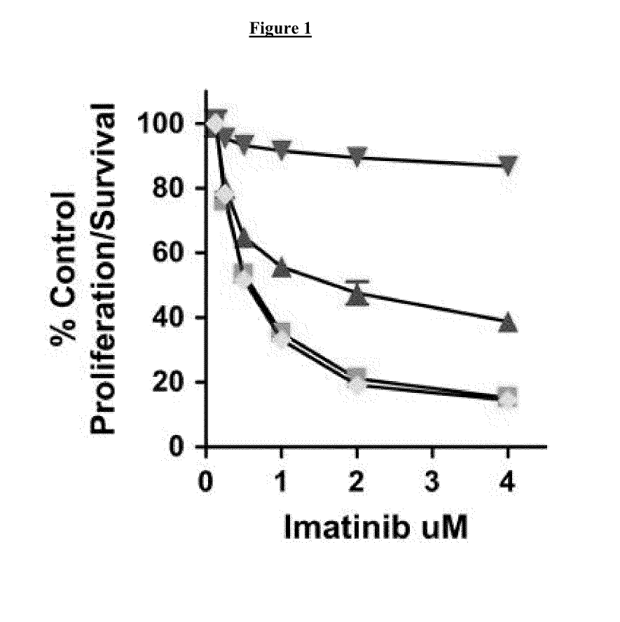

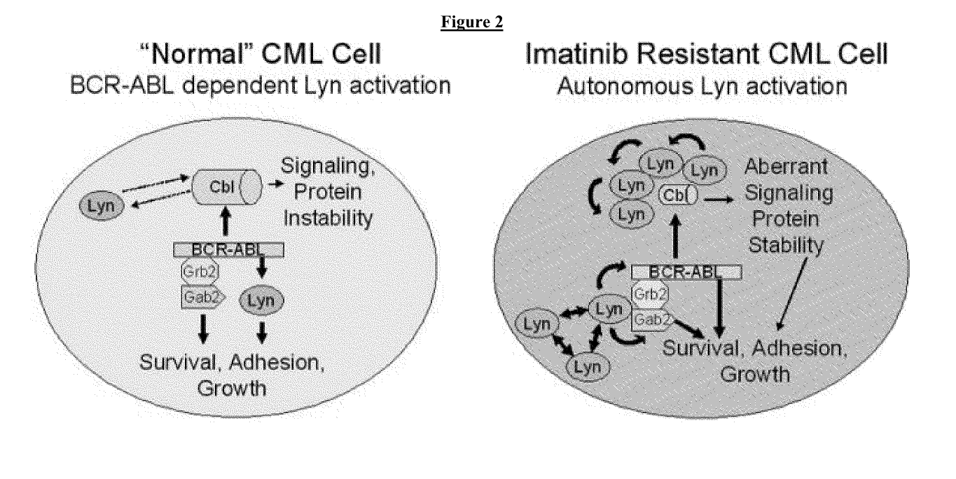

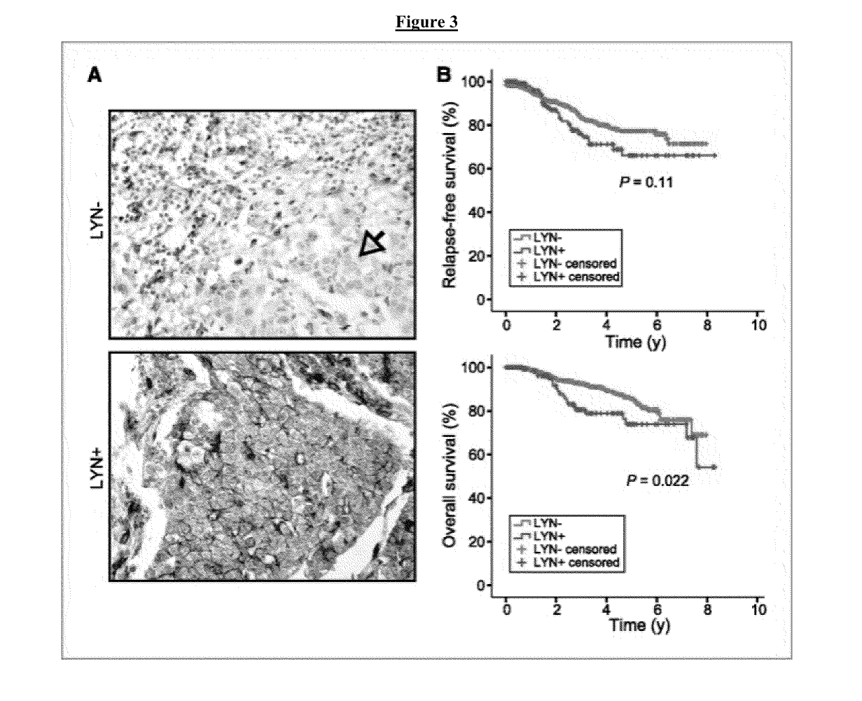

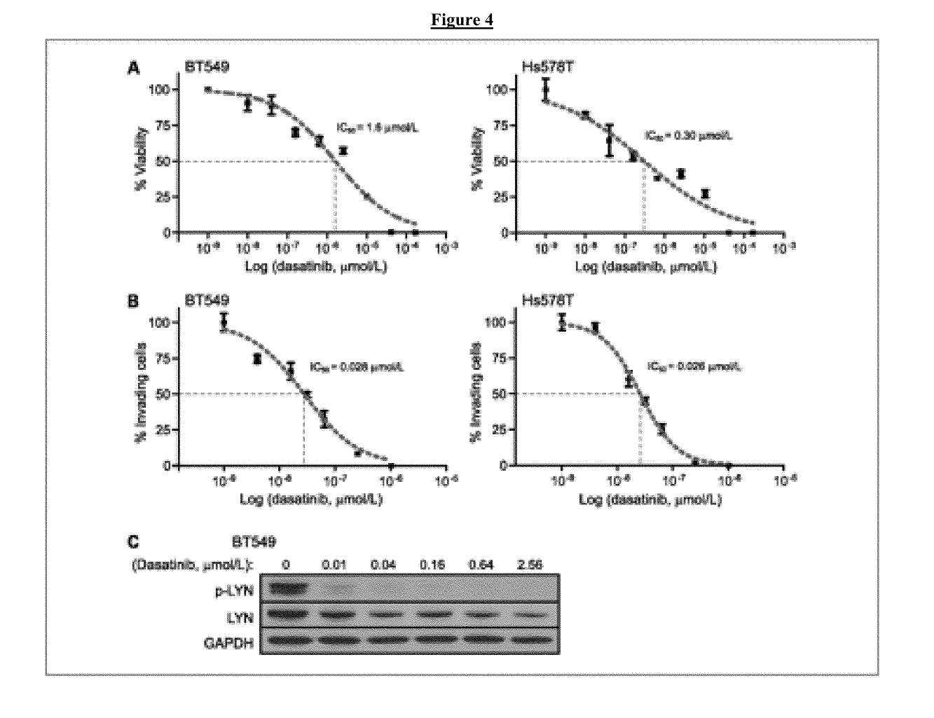

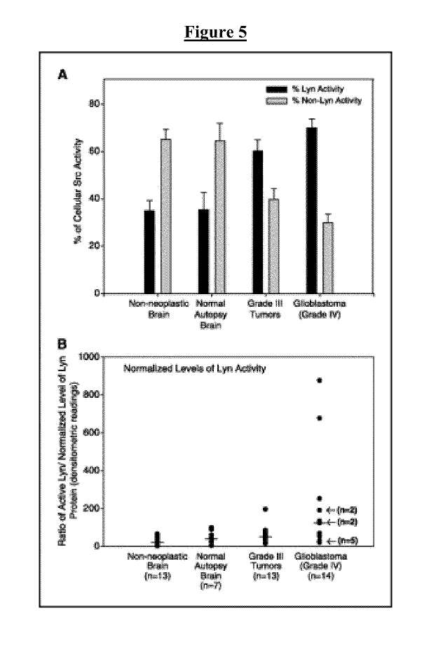

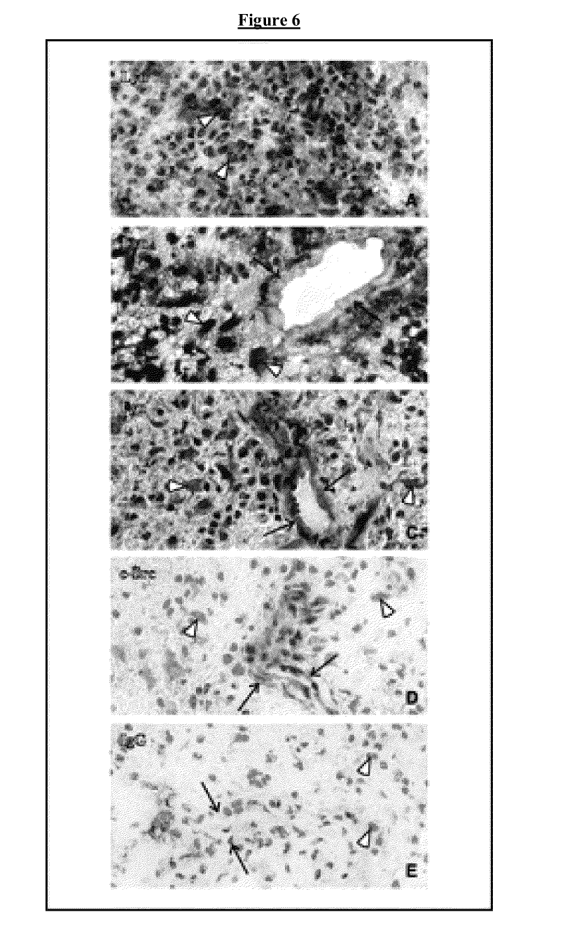

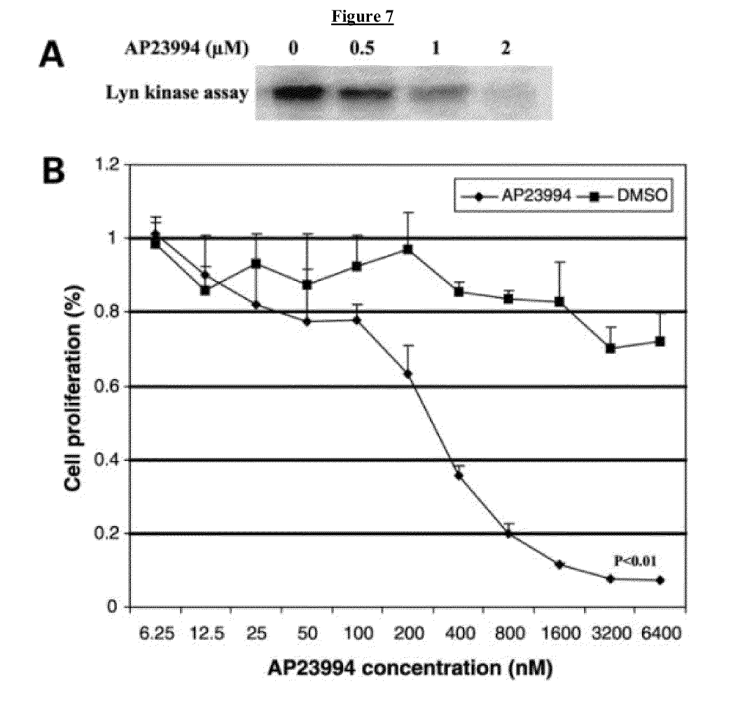

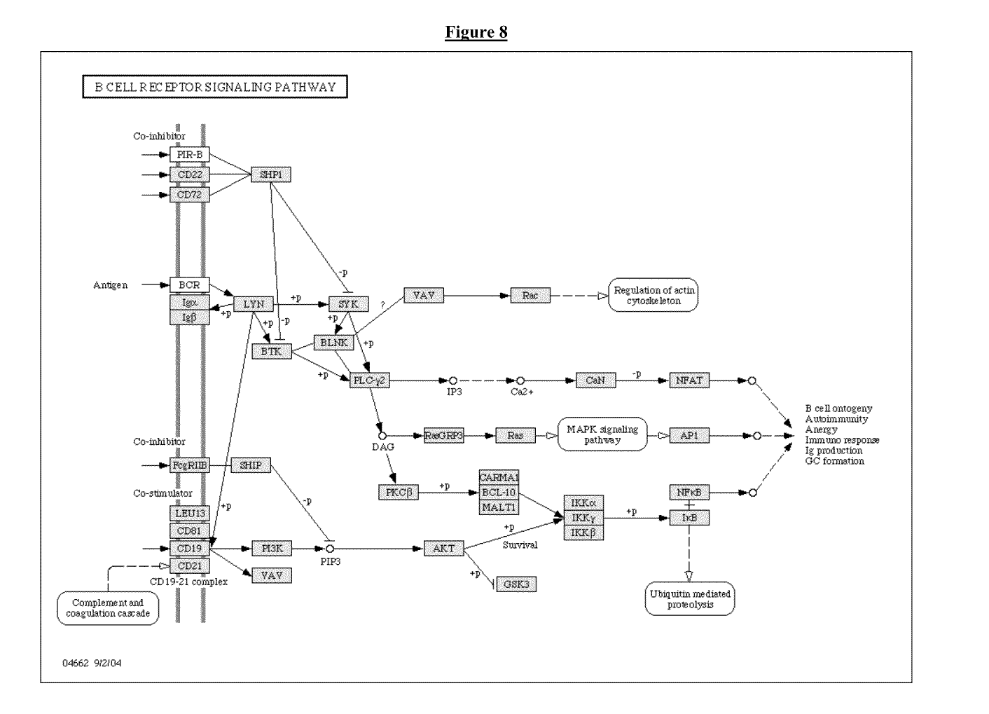

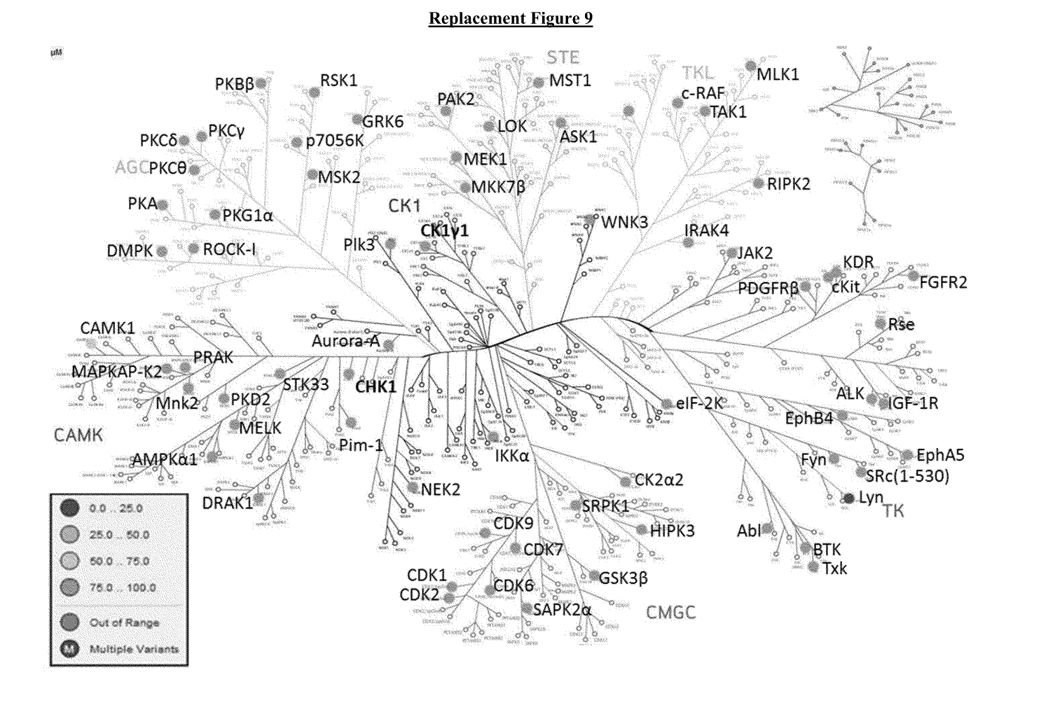

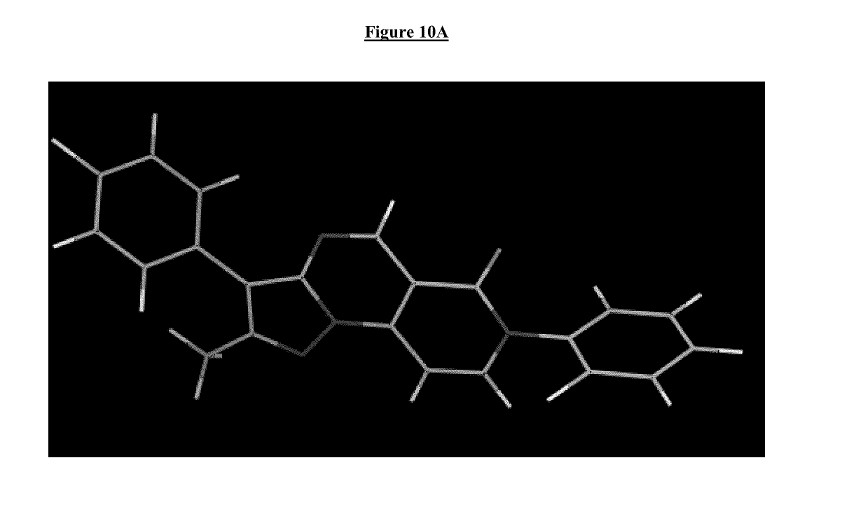

### DETAILED DESCRIPTION OF THE INVENTION

The present invention relates to the unexpected discovery of novel compounds that inhibit the activity of LYN kinase in vitro and in vivo. The compounds of the invention are thus useful for treating, ameliorating or preventing cancer in a subject. In one embodiment, the cancer is dependent on LYN activity, whereby administration of the compounds of the invention to a subject with the cancer inhibits LYN kinase activity in the subject and treats, ameliorates or prevents the cancer in the subject. In another embodiment, the cancer is selected from the group consisting of CML, B-CLL, glioblastoma, breast cancer, Ewing's sarcoma and prostate cancer. In yet another embodiment, the CML cancer is resistant to 4-[(4-methylpiperazin-1-yl)methyl]-N-[4-methyl-3-[(4-pyridin-3-ylpyrimidin-2-yl)amino]phenyl]benzamide (imatinib).

### DEFINITIONS

As used herein, each of the following terms has the meaning associated with it in this section.

Unless defined otherwise, all technical and scientific terms used herein generally have the same meaning as commonly understood by one of ordinary skill in the art to which this invention belongs. Generally, the nomenclature used herein and the laboratory procedures in cell culture, molecular genetics, analytical chemistry, organic chemistry, and nucleic acid chemistry and hybridization are those well-known and commonly employed in the art. Standard techniques or modifications thereof are used for chemical syntheses and chemical analyses.

The articles “a” and “an” are used herein to refer to one or to more than one (i.e. to at least one) of the grammatical object of the article. By way of example, “an element” means one element or more than one element.

As used herein, the term “about” will be understood by persons of ordinary skill in the art and will vary to some extent on the context in which it is used. As used herein when referring to a measurable value such as an amount, a temporal duration, and the like, the term “about” is meant to encompass variations of ±20% or ±10%, more preferably ±5%, even more preferably ±1%, and still more preferably ±0.1% from the specified value, as such variations are appropriate to perform the disclosed methods.

An “amino acid” as used herein is meant to include both natural and synthetic amino acids, and both D and L amino acids. “Standard amino acid” means any of the twenty L-amino acids commonly found in naturally occurring peptides. “Nonstandard amino acid residues” means any amino acid, other than the standard amino acids, regardless of whether it is prepared synthetically or derived from a natural source. As used herein, “synthetic amino acid” also encompasses chemically modified amino acids, including but not limited to salts, amino acid derivatives (such as amides), and substitutions. Amino acids contained within the peptides, and particularly at the carboxy- or amino-terminus, can be modified by methylation, amidation, acetylation or substitution with other chemical groups which can change a peptide's circulating half-life without adversely affecting activity of the peptide. Additionally, a disulfide linkage may be present or absent in the peptides.

As used herein, the terms “protein”, “peptide” and “polypeptide” are used interchangeably, and refer to a compound comprised of amino acid residues covalently linked by peptide bonds. The term “peptide bond” means a covalent amide linkage formed by loss of a molecule of water between the carboxyl group of one amino acid and the amino group of a second amino acid. A protein or peptide must contain at least two amino acids, and no limitation is placed on the maximum number of amino acids that may comprise the sequence of a protein or peptide. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Proteins” include, for example, biologically active fragments, substantially homologous proteins, oligopeptides, homodimers, heterodimers, variants of proteins, modified proteins, derivatives, analogs, and fusion proteins, among others. The proteins include natural proteins, recombinant proteins, synthetic proteins, or a combination thereof. A protein may be a receptor or a non-receptor.

As used herein, the term “fragment,” as applied to a protein or peptide, refers to a subsequence of a larger protein or peptide. A “fragment” of a protein or peptide can be at least about 20 amino acids in length; for example at least about 50 amino acids in length; at least about 100 amino acids in length, at least about 200 amino acids in length, at least about 300 amino acids in length, and at least about 400 amino acids in length (and any integer value in between).

By the term “specifically binds,” as used herein, is meant a molecule, such as an antibody, which recognizes and binds to another molecule or feature, but does not substantially recognize or bind other molecules or features in a sample.

The phrase “inhibit,” as used herein, means to reduce a molecule, a reaction, an interaction, a gene, an mRNA, and/or a protein's expression, stability, function or activity by a measurable amount or to prevent entirely. Inhibitors are compounds that, e.g., bind to, partially or totally block stimulation, decrease, prevent, delay activation, inactivate, desensitize, or down regulate a protein, a gene, and an mRNA stability, expression, function and activity, e.g., antagonists.

“Effective amount” or “therapeutically effective amount” are used interchangeably herein, and refer to an amount of a compound, formulation, material, or composition, as described herein effective to achieve a particular biological result. Such results may include, but are not limited to, the treatment of a disease or condition as determined by any means suitable in the art.

As used herein, the term “pharmaceutical composition” refers to a mixture of at least one compound of the invention with other chemical components, such as carriers, stabilizers, diluents, dispersing agents, suspending agents, thickening agents, and/or excipients. The pharmaceutical composition facilitates administration of the compound to an organism. Multiple techniques of administering a compound exist in the art including, but not limited to, intravenous, oral, aerosol, parenteral, ophthalmic, pulmonary and topical administration.

“Pharmaceutically acceptable” refers to those properties and/or substances which are acceptable to the patient from a pharmacological/toxicological point of view and to the manufacturing pharmaceutical chemist from a physical/chemical point of view regarding composition, formulation, stability, patient acceptance and bioavailability. “Pharmaceutically acceptable carrier” refers to a medium that does not interfere with the effectiveness of the biological activity of the active ingredient(s) and is not toxic to the host to which it is administered.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the invention within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the invention, and are physiologically acceptable to the patient. Supplementary active compounds may also be incorporated into the compositions. The “pharmaceutically acceptable carrier” may further include a pharmaceutically acceptable salt of the compound useful within the invention. Other additional ingredients that may be included in the pharmaceutical compositions used in the practice of the invention are known in the art and described, for example in Remington's Pharmaceutical Sciences (Genaro, Ed., Mack Publishing Co., 1985, Easton, Pa.), which is incorporated herein by reference.

As used herein, the term “salt” embraces addition salts of free acids or free bases that are compounds useful within the invention. Suitable acid addition salts may be prepared from an inorganic acid or from an organic acid. Examples of inorganic acids include hydrochloric, hydrobromic, hydriodic, nitric, carbonic, sulfuric, phosphoric acids, perchloric and tetrafluoroboronic acids. Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, β-hydroxybutyric, salicylic, galactaric and galacturonic acid. Suitable base addition salts of compounds useful within the invention include, for example, metallic salts including alkali metal, alkaline earth metal and transition metal salts such as, for example, lithium, calcium, magnesium, potassium, sodium and zinc salts. Acceptable base addition salts also include organic salts made from basic amines such as, for example, N,N′-dibenzylethylenediamine, chloroprocaine, choline, diethanolamine, ethylenediamine, meglumine (N-methyl-glutamine) and procaine. All of these salts may be prepared by conventional means from the corresponding free base compound by reacting, for example, the appropriate acid or base with the corresponding free base.

As used herein, the term “alkyl,” by itself or as part of another substituent means, unless otherwise stated, a straight or branched chain hydrocarbon having the number of carbon atoms designated (i.e. C₁₋₆ means one to six carbon atoms) and includes straight, branched chain, or cyclic substituent groups. Examples include methyl, ethyl, propyl, isopropyl, butyl, isobutyl, tert-butyl, pentyl, neopentyl, hexyl, and cyclopropylmethyl. Most preferred is (C₁-C₆)alkyl, particularly ethyl, methyl, isopropyl, isobutyl, n-pentyl, n-hexyl and cyclopropylmethyl.

As used herein, the term “substituted alkyl” means alkyl, as defined above, substituted by one, two or three substituents selected from the group consisting of halogen, —OH, alkoxy, —NH₂, —N(CH₃)₂, —C(═O)OH, trifluoromethyl, —C≡N, —C(═O)O(C₁-C₄)alkyl, —C(═O)NH₂, —SO₂NH₂, —C(═NH)NH₂, and —NO₂, preferably containing one or two substituents selected from halogen, —OH, alkoxy, —NH₂, trifluoromethyl, —N(CH₃)₂, and —C(═O)OH, more preferably selected from halogen, alkoxy and —OH. Examples of substituted alkyls include, but are not limited to, 2,2-difluoropropyl, 2-carboxycyclopentyl and 3-chloropropyl.

As used herein, the term “alkoxy” employed alone or in combination with other terms means, unless otherwise stated, an alkyl group having the designated number of carbon atoms, as defined above, connected to the rest of the molecule via an oxygen atom, such as, for example, methoxy, ethoxy, 1-propoxy, 2-propoxy (isopropoxy) and the higher homologs and isomers. Preferred are (C₁-C₃) alkoxy, particularly ethoxy and methoxy.

As used herein, the term “halo” or “halogen” alone or as part of another substituent means, unless otherwise stated, a fluorine, chlorine, bromine, or iodine atom, preferably, fluorine, chlorine, or bromine, more preferably, fluorine or chlorine.

As used herein, the term “heteroalkyl” by itself or in combination with another term means, unless otherwise stated, a stable straight or branched chain alkyl group consisting of the stated number of carbon atoms and one or two heteroatoms selected from the group consisting of O, N, and S, and wherein the nitrogen and sulfur atoms may be optionally oxidized and the nitrogen heteroatom may be optionally quaternized. The heteroatom(s) may be placed at any position of the heteroalkyl group, including between the rest of the heteroalkyl group and the fragment to which it is attached, as well as attached to the most distal carbon atom in the heteroalkyl group. Examples include: —O—CH₂—CH₂—CH₃, —CH₂—CH₂—CH₂—OH, —CH₂—CH₂—NH—CH₃, —CH₂—S—CH₂—CH₃, and —CH₂CH₂—S(═O)—CH₃. Up to two heteroatoms may be consecutive, such as, for example, —CH₂—NH—OCH₃, or —CH₂—CH₂—S—S—CH₃

As used herein, the term “aromatic” refers to a carbocycle or heterocycle with one or more polyunsaturated rings and having aromatic character, i.e. having (4n+2) delocalized π (pi) electrons, where n is an integer.

As used herein, the term “aryl,” employed alone or in combination with other terms, means, unless otherwise stated, a carbocyclic aromatic system containing one or more rings (typically one, two or three rings) wherein such rings may be attached together in a pendent manner, such as a biphenyl, or may be fused, such as naphthalene. Examples include phenyl, anthracyl, and naphthyl. Preferred are phenyl and naphthyl, most preferred is phenyl.

As used herein, the term “aryl-(C₁-C₃)alkyl” means a functional group wherein a one to three carbon alkylene chain is attached to an aryl group, e.g., —CH₂CH₂-phenyl. Preferred is aryl-CH₂— and aryl-CH(CH₃)—. The term “substituted aryl-(C₁-C₃)alkyl” means an aryl-(C₁-C₃)alkyl functional group in which the aryl group is substituted. Preferred is substituted aryl(CH₂)—. Similarly, the term “heteroaryl-(C₁-C₃)alkyl” means a functional group wherein a one to three carbon alkylene chain is attached to a heteroaryl group, e.g., —CH₂CH₂-pyridyl. Preferred is heteroaryl-(CH₂)—. The term “substituted heteroaryl-(C₁-C₃)alkyl” means a heteroaryl-(C₁-C₃)alkyl functional group in which the heteroaryl group is substituted. Preferred is substituted heteroaryl-(CH₂)—.

As used herein, the term “heterocycle” or “heterocyclyl” or “heterocyclic” by itself or as part of another substituent means, unless otherwise stated, an unsubstituted or substituted, stable, mono- or multi-cyclic heterocyclic ring system that consists of carbon atoms and at least one heteroatom selected from the group consisting of N, O, and S, and wherein the nitrogen and sulfur heteroatoms may be optionally oxidized, and the nitrogen atom may be optionally quaternized. The heterocyclic system may be attached, unless otherwise stated, at any heteroatom or carbon atom that affords a stable structure. A heterocycle may be aromatic or non-aromatic in nature. In one embodiment, the heterocycle is a heteroaryl.

As used herein, the term “heteroaryl” or “heteroaromatic” refers to a heterocycle having aromatic character. A polycyclic heteroaryl may include one or more rings that are partially saturated. Examples include tetrahydroquinoline and 2,3-dihydrobenzofuryl.

Examples of non-aromatic heterocycles include monocyclic groups such as aziridine, oxirane, thiirane, azetidine, oxetane, thietane, pyrrolidine, pyrroline, imidazoline, pyrazolidine, dioxolane, sulfolane, 2,3-dihydrofuran, 2,5-dihydrofuran, tetrahydrofuran, thiophane, piperidine, 1,2,3,6-tetrahydropyridine, 1,4-dihydropyridine, piperazine, morpholine, thiomorpholine, pyran, 2,3-dihydropyran, tetrahydropyran, 1,4-dioxane, 1,3-dioxane, homopiperazine, homopiperidine, 1,3-dioxepane, 4,7-dihydro-1,3-dioxepin and hexamethyleneoxide.

Examples of heteroaryl groups include pyridyl, pyrazinyl, pyrimidinyl (particularly 2- and 4-pyrimidinyl), pyridazinyl, thienyl, furyl, pyrrolyl (particularly 2-pyrrolyl), imidazolyl, thiazolyl, oxazolyl, pyrazolyl (particularly 3- and 5-pyrazolyl), isothiazolyl, 1,2,3-triazolyl, 1,2,4-triazolyl, 1,3,4-triazolyl, tetrazolyl, 1,2,3-thiadiazolyl, 1,2,3-oxadiazolyl, 1,3,4-thiadiazolyl and 1,3,4-oxadiazolyl.

Examples of polycyclic heterocycles include indolyl (particularly 3-, 4-, 5-, 6- and 7-indolyl), indolinyl, quinolyl, tetrahydroquinolyl, isoquinolyl (particularly 1- and 5-isoquinolyl), 1,2,3,4-tetrahydroisoquinolyl, cinnolinyl, quinoxalinyl (particularly l- and 5-quinoxalinyl), quinazolinyl, phthalazinyl, 1,8-naphthyridinyl, 1,4-benzodioxanyl, coumarin, dihydrocoumarin, 1,5-naphthyridinyl, benzofuryl (particularly 3-, 4-, 5-, 6- and 7-benzofuryl), 2,3-dihydrobenzofuryl, 1,2-benzisoxazolyl, benzothienyl (particularly 3-, 4-, 5-, 6-, and 7-benzothienyl), benzoxazolyl, benzothiazolyl (particularly 2-benzothiazolyl and 5-benzothiazolyl), purinyl, benzimidazolyl (particularly 2-benzimidazolyl), benztriazolyl, thioxanthinyl, carbazolyl, carbolinyl, acridinyl, pyrrolizidinyl, and quinolizidinyl.

The aforementioned listing of heterocyclyl and heteroaryl moieties is intended to be representative and not limiting.

As used herein, the term “substituted” means that an atom or group of atoms has replaced hydrogen as the substituent attached to another group.

For aryl, aryl-(C₁-C₃)alkyl and heterocyclyl groups, the term “substituted” as applied to the rings of these groups refers to any level of substitution, namely mono-, di-, tri-, tetra-, or penta-substitution, where such substitution is permitted. The substituents are independently selected, and substitution may be at any chemically accessible position. In one embodiment, the substituents vary in number between one and four. In another embodiment, the substituents vary in number between one and three. In yet another embodiment, the substituents vary in number between one and two. In yet another embodiment, the substituents are independently selected from the group consisting of C₁₋₆ alkyl, —OH, C₁₋₆ alkoxy, halo, amino, acetamido and nitro. In yet another embodiment, the substituents are independently selected from the group consisting of C₁₋₆ alkyl, C₁₋₆ alkoxy, halo, acetamido, and nitro. As used herein, where a substituent is an alkyl or alkoxy group, the carbon chain may be branched, straight or cyclic, with straight being preferred.

An “individual”, “patient” or “subject”, as that term is used herein, includes a member of any animal species including, but are not limited to, birds, humans and other primates, and other mammals including commercially relevant mammals such as cattle, pigs, horses, sheep, cats, and dogs. Preferably, the subject is a human.

The term to “treat,” as used herein, means reducing the frequency with which symptoms are experienced by a subject or administering an agent or compound to reduce the frequency and/or severity with which symptoms are experienced. As used herein, “alleviate” is used interchangeably with the term “treat.” The term “therapeutic” as used herein means a treatment and/or prophylaxis. A therapeutic effect is obtained by suppression, remission, or eradication of UVR-induced skin damage.

As used herein, “treating a disease, disorder or condition” means reducing the frequency or severity with which a symptom of the disease, disorder or condition is experienced by a subject. Treating a disease, disorder or condition may or may not include complete eradication or elimination of the symptom.

“Instructional material,” as that term is used herein, includes a publication, a recording, a diagram, or any other medium of expression which can be used to communicate the usefulness of the composition and/or compound of the invention in a kit. The instructional material of the kit may, for example, be affixed to a container that contains the compound and/or composition of the invention or be shipped together with a container which contains the compound and/or composition. Alternatively, the instructional material may be shipped separately from the container with the intention that the recipient uses the instructional material and the compound cooperatively. Delivery of the instructional material may be, for example, by physical delivery of the publication or other medium of expression communicating the usefulness of the kit, or may alternatively be achieved by electronic transmission, for example by means of a computer, such as by electronic mail, or download from a website.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Compounds

The compounds useful within the invention may be synthesized using techniques well-known in the art of organic synthesis or obtained from commercial sources.

In one aspect, the compound of the invention has the formula (I):

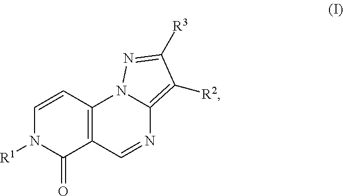

or a salt thereof, wherein:

R¹ and R⁶ are independently unsubstituted or substituted alkyl, unsubstituted or substituted aryl, or unsubstituted or substituted heteroaryl; and

R³ is H or C₁-C₆ alkyl;

with the proviso that the compound of formula (I) is not 7-(2-fluorophenyl)-3-(4-fluorophenyl)-2-methylpyrazolo[1,5-a]pyrido[3,4-e]pyrimidin-6(7H)-one; 3-(4-chlorophenyl)-7-(2-fluorophenyl)-2-methylpyrazolo[1,5-a]pyrido[3,4-e]pyrimidin-6(7H)-one: 7-(2-fluorophenyl)-2-methyl-3-phenylpyrazolo[1,5-a]pyrido[3,4-e]pyrimidin-6(7H)-one; 7-(3-fluorophenyl)-2-methyl-3-phenylpyrazolo[1,5-a]pyrido[3,4-e]pyrimidin-6(7H)-one; 7-(3-chloro-4-fluorophenyl)-2-methyl-3-phenylpyrazolo[1,5-a]pyrido[3,4-e]pyrimidin-6(7H)-one; 7-(3,4-difluorophenyl)-2-methyl-3-phenylpyrazolo[1,5-a]pyrido[3,4-e]pyrimidin-6(7H)-one; 7-(3-chlorophenyl)-3-(4-fluorophenyl)-2-methylpyrazolo[1,5-a]pyrido[3,4-e]pyrimidin-6(7H)-one; 7-(2-chlorophenyl)-3-(4-fluorophenyl)-2-methylpyrazolo[1,5-a]pyrido[3,4-e]pyrimidin-6(7H)-one; 3-(4-fluorophenyl)-7-(2-methoxyphenyl)-2-methylpyrazolo[1,5-a]pyrido[3,4-e]pyrimidin-6(7H)-one; 2-(3-(4-fluorophenyl)-2-methyl-6-oxopyrazolo[1,5-a]pyrido[3,4-e]pyrimidin-7(6H)-yl)benzonitrile; 2-ethyl-3-(4-fluorophenyl)-7-phenylpyrazolo[1,5-a]pyrido[3,4-e]pyrimidin-6(7H)-one; or a salt thereof.

In one embodiment, R¹ and R² are independently unsubstituted or substituted aryl, or unsubstituted or substituted heteroaryl. In another embodiment, R³ is H, methyl or ethyl.

### Salts

The compounds described herein may form salts with acids or bases, and such salts are included in the present invention. In one embodiment, the salts are pharmaceutically acceptable salts. The term “salts” embraces addition salts of free acids or free bases that are compounds of the invention. The term “pharmaceutically acceptable salt” refers to salts that possess toxicity profiles within a range that affords utility in pharmaceutical applications. Pharmaceutically unacceptable salts may nonetheless possess properties such as high crystallinity, which have utility in the practice of the present invention, such as for example utility in process of synthesis, purification or formulation of compounds of the invention.

Suitable pharmaceutically acceptable acid addition salts may be prepared from an inorganic acid or from an organic acid. Examples of inorganic acids include hydrochloric, hydrobromic, hydriodic, nitric, carbonic, sulfuric, and phosphoric acids. Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, β-hydroxybutyric, salicylic, galactaric and galacturonic acid.

Examples of pharmaceutically unacceptable acid addition salts include, for example, perchlorates and tetrafluoroborates. Suitable pharmaceutically acceptable base addition salts of compounds of the invention include, for example, metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N′-dibenzylethylene-diamine, chloroprocaine, choline, diethanolamine, ethylenediamine, meglumine (N-methylglucamine) and procaine. Examples of pharmaceutically unacceptable base addition salts include lithium salts and cyanate salts. All of these salts may be prepared from the corresponding compound by reacting, for example, the appropriate acid or base

### LYN Kinase Inhibitors: Small Molecules

A small molecule inhibitor of LYN kinase may be obtained using standard methods known to the skilled artisan. Such methods include chemical organic synthesis or biological means. Biological means include purification from a biological source, recombinant synthesis and in vitro translation systems, using methods well known in the art.

Identification of a small molecule that inhibits LYN kinase activity may be achieved using the ChemVassa methodology. Variations on the structure of the identified hits may be performed using directed synthetic methods or combinatorial methods. Combinatorial libraries of molecularly diverse chemical compounds potentially useful in treating a variety of diseases and conditions are well known in the art as are method of making the libraries. The method may use a variety of techniques well-known to the skilled artisan including solid phase synthesis, solution methods, parallel synthesis of single compounds, synthesis of chemical mixtures, rigid core structures, flexible linear sequences, deconvolution strategies, tagging techniques, and generating unbiased molecular landscapes for lead discovery vs. biased structures for lead development.

In a general method for small library synthesis, an activated core molecule is condensed with a number of building blocks, resulting in a combinatorial library of covalently linked, core-building block ensembles. The shape and rigidity of the core determines the orientation of the building blocks in shape space. The libraries can be biased by changing the core, linkage, or building blocks to target a characterized biological structure (“focused libraries”) or synthesized with less structural bias using flexible cores.

### METHODS OF THE INVENTION

The invention includes a method of inhibiting LYN kinase activity in a subject in need thereof. The method comprises administering to the subject a pharmaceutical composition comprising a pharmaceutically acceptable carrier and a therapeutically effective amount of a LYN kinase inhibitor, where the inhibitor is a compound of formula (II):

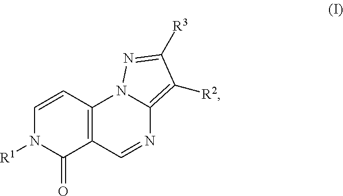

or a salt thereof, wherein:
R¹ and R⁶ are independently unsubstituted or substituted alkyl, unsubstituted or substituted aryl, or unsubstituted or substituted heteroaryl; and
R³ is H or C₁-C₆ alkyl.

In one embodiment, the compound of formula (II) is selected from the group consisting of:
7-(2-fluorophenyl)-3-(4-fluorophenyl)-2-methylpyrazolo[1,5-a]pyrido[3,4-e]pyrimidin-6(7H)-one:

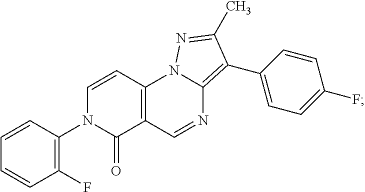 [SMILES: Cc1nn2c(ncc3c(=O)n(-c4ccccc4F)ccc32)c1-c1ccc(F)cc1]

3-(4-chlorophenyl)-7-(2-fluorophenyl)-2-methylpyrazolo[1,5-a]pyrido[3,4-e]pyrimidin-6(7H)-one:

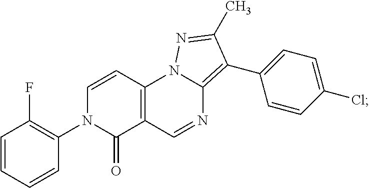 [SMILES: Cc1nn2c(ncc3c(=O)n(-c4ccccc4F)ccc32)c1-c1ccc(Cl)cc1]

7-(2-fluorophenyl)-2-methyl-3-phenylpyrazolo[1,5-a]pyrido[3,4-e]pyrimidin-6(7H)-one:

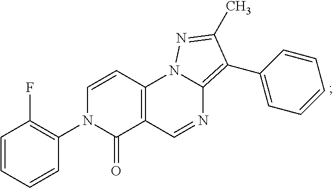 [SMILES: Cc1nn2c(ncc3c(=O)n(-c4ccccc4F)ccc32)c1-c1ccccc1]

7-(3-fluorophenyl)-2-methyl-3-phenylpyrazolo[1,5-a]pyrido[3,4-e]pyrimidin-6(7H)-one:

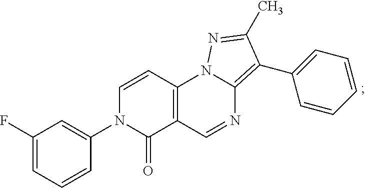 [SMILES: Cc1nn2c(ncc3c(=O)n(-c4cccc(F)c4)ccc32)c1-c1ccccc1]

7-(3-chloro-4-fluorophenyl)-2-methyl-3-phenylpyrazolo[1,5-a]pyrido[3,4-e]pyrimidin-6(7H)-one:

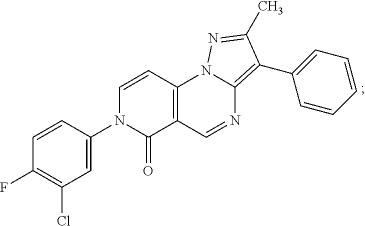 [SMILES: Cc1nn2c(ncc3c(=O)n(-c4ccc(F)c(Cl)c4)ccc32)c1-c1ccccc1]

7-(3,4-difluorophenyl)-2-methyl-3-phenylpyrazolo[1,5-a]pyrido[3,4-e]pyrimidin-6(7H)-one:

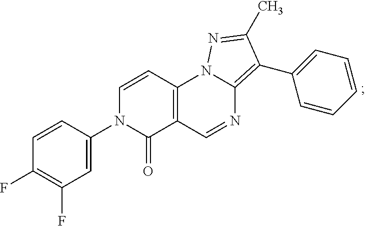 [SMILES: Cc1nn2c(ncc3c(=O)n(-c4ccc(F)c(F)c4)ccc32)c1-c1ccccc1]

7-(3-chlorophenyl)-3-(4-fluorophenyl)-2-methylpyrazolo[1,5-a]pyrido[3,4-e]pyrimidin-6(7H)-one:

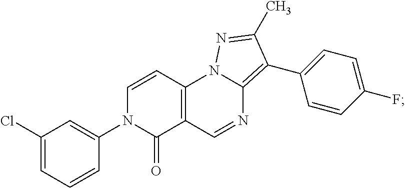 [SMILES: Cc1nn2c(ncc3c(=O)n(-c4cccc(Cl)c4)ccc32)c1-c1ccc(F)cc1]

7-(2-chlorophenyl)-3-(4-fluorophenyl)-2-methylpyrazolo[1,5-a]pyrido[3,4-e]pyrimidin-6(7H)-one:

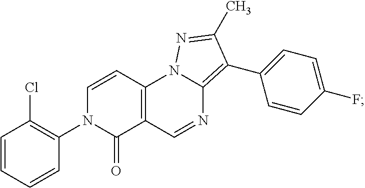 [SMILES: Cc1nn2c(ncc3c(=O)n(-c4ccccc4Cl)ccc32)c1-c1ccc(F)cc1]

3-(4-fluorophenyl)-7-(2-methoxyphenyl)-2-methylpyrazolo[1,5-a]pyrido[3,4-e]pyrimidin-6(7H)-one:

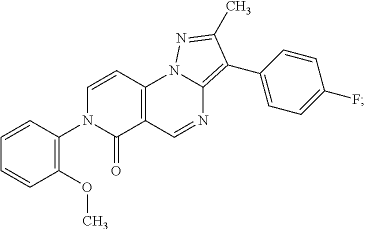 [SMILES: COc1ccccc1-n1ccc2c(cnc3c(-c4ccc(F)cc4)c(C)nn32)c1=O]

2-(3-(4-fluorophenyl)-2-methyl-6-oxopyrazolo[1,5-a]pyrido[3,4-e]pyrimidin-7(6H)-yl)benzonitrile:

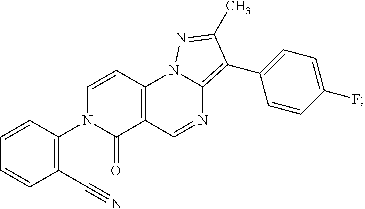 [SMILES: Cc1nn2c(ncc3c(=O)n(-c4ccccc4C#N)ccc32)c1-c1ccc(F)cc1]

2-ethyl-3-(4-fluorophenyl)-7-phenylpyrazolo[1,5-a]pyrido[3,4-e]pyrimidin-6(7H)-one:

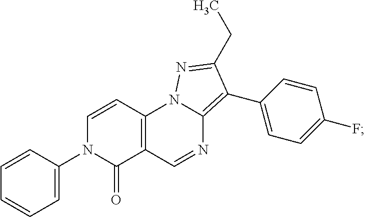 [SMILES: CCc1nn2c(ncc3c(=O)n(-c4ccccc4)ccc32)c1-c1ccc(F)cc1]

and a salt thereof. In another embodiment, the subject is human.

The invention also includes a method of treating, ameliorating or preventing cancer in a subject in need thereof, wherein the cancer is dependent on LYN kinase activity. The method comprises administering to the subject a pharmaceutical composition comprising a pharmaceutically acceptable carrier and a therapeutically effective amount of a LYN kinase inhibitor, whereby the administration of the composition to the subject inhibits LYN kinase activity, wherein the inhibitor is a compound of formula (II):

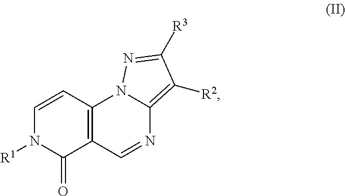

or a salt thereof, wherein:
R¹ and R⁶ are independently unsubstituted or substituted alkyl, unsubstituted or substituted aryl, or unsubstituted or substituted heteroaryl; and
R³ is H or C₁-C₆ alkyl.

In one embodiment, the cancer is selected from the group consisting of CML, B-CLL, glioblastoma, breast cancer, Ewing's sarcoma and prostate cancer. In another embodiment, the CML cancer is resistant to treatment with 4-[(4-methylpiperazin-1-yl)methyl]-N-[4-methyl-3-[(4-pyridin-3-ylpyrimidin-2-yl)amino]phenyl]benzamide).

In one embodiment, the compound of formula (II) is selected from the group consisting of:
7-(2-fluorophenyl)-3-(4-fluorophenyl)-2-methylpyrazolo[1,5-a]pyrido[3,4-e]pyrimidin-6(7H)-one:

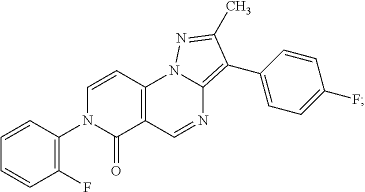 [SMILES: Cc1nn2c(ncc3c(=O)n(-c4ccccc4F)ccc32)c1-c1ccc(F)cc1]

3-(4-chlorophenyl)-7-(2-fluorophenyl)-2-methylpyrazolo[1,5-a]pyrido[3,4-e]pyrimidin-6(7H)-one:

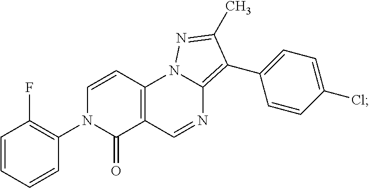 [SMILES: Cc1nn2c(ncc3c(=O)n(-c4ccccc4F)ccc32)c1-c1ccc(Cl)cc1]

7-(2-fluorophenyl)-2-methyl-3-phenylpyrazolo[1,5-a]pyrido[3,4-e]pyrimidin-6(7H)-one:

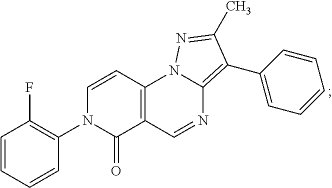 [SMILES: Cc1nn2c(ncc3c(=O)n(-c4ccccc4F)ccc32)c1-c1ccccc1]

7-(3-fluorophenyl)-2-methyl-3-phenylpyrazolo[1,5-a]pyrido[3,4-e]pyrimidin-6(7H)-one:

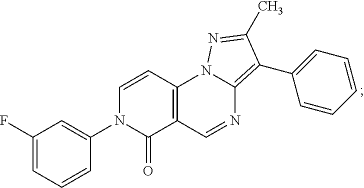 [SMILES: Cc1nn2c(ncc3c(=O)n(-c4cccc(F)c4)ccc32)c1-c1ccccc1]

7-(3-chloro-4-fluorophenyl)-2-methyl-3-phenylpyrazolo[1,5-a]pyrido[3,4-e]pyrimidin-6(7H)-one:

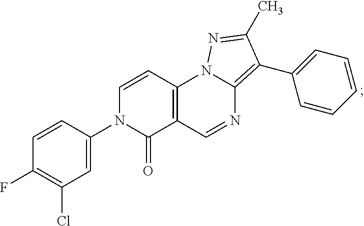 [SMILES: Cc1nn2c(ncc3c(=O)n(-c4ccc(F)c(Cl)c4)ccc32)c1-c1ccccc1]

7-(3,4-difluorophenyl)-2-methyl-3-phenylpyrazolo[1,5-a]pyrido[3,4-e]pyrimidin-6(7H)-one:

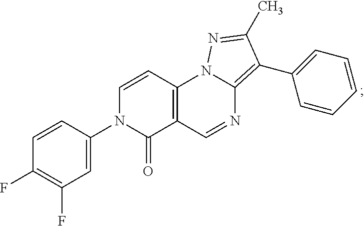 [SMILES: Cc1nn2c(ncc3c(=O)n(-c4ccc(F)c(F)c4)ccc32)c1-c1ccccc1]

7-(3-chlorophenyl)-3-(4-fluorophenyl)-2-methylpyrazolo[1,5-a]pyrido[3,4-e]pyrimidin-6(7H)-one:

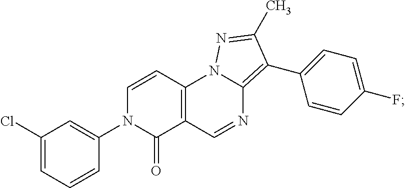 [SMILES: Cc1nn2c(ncc3c(=O)n(-c4cccc(Cl)c4)ccc32)c1-c1ccc(F)cc1]

7-(2-chlorophenyl)-3-(4-fluorophenyl)-2-methylpyrazolo[1,5-a]pyrido[3,4-e]pyrimidin-6(7H)-one:

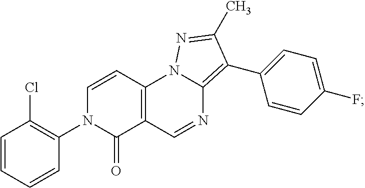 [SMILES: Cc1nn2c(ncc3c(=O)n(-c4ccccc4Cl)ccc32)c1-c1ccc(F)cc1]

3-(4-fluorophenyl)-7-(2-methoxyphenyl)-2-methylpyrazolo[1,5-a]pyrido[3,4-e]pyrimidin-6(7H)-one:

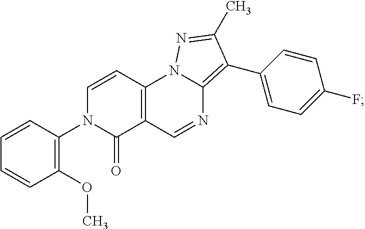 [SMILES: COc1ccccc1-n1ccc2c(cnc3c(-c4ccc(F)cc4)c(C)nn32)c1=O]

2-(3-(4-fluorophenyl)-2-methyl-6-oxopyrazolo[1,5-a]pyrido[3,4-e]pyrimidin-7(6H)-yl)benzonitrile:

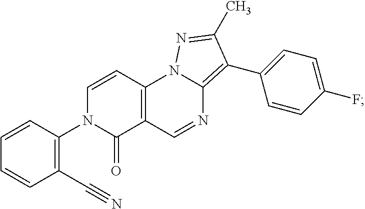 [SMILES: Cc1nn2c(ncc3c(=O)n(-c4ccccc4C#N)ccc32)c1-c1ccc(F)cc1]

2-ethyl-3-(4-fluorophenyl)-7-phenylpyrazolo[1,5-a]pyrido[3,4-e]pyrimidin-6(7H)-one:

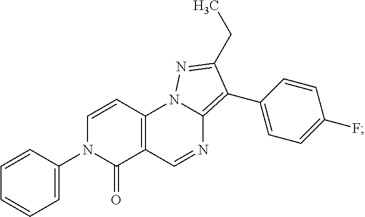 [SMILES: CCc1nn2c(ncc3c(=O)n(-c4ccccc4)ccc32)c1-c1ccc(F)cc1]

and a salt thereof. In another embodiment, the subject is human.

### Pharmaceutical Compositions and Therapies

Administration of a LYN kinase inhibitor in a method of treatment may be achieved in a number of different ways, using methods known in the art. The therapeutic and prophylactic methods of the invention thus encompass the use of pharmaceutical compositions comprising the LYN kinase inhibitors of the invention to practice the methods of the invention. The pharmaceutical compositions useful for practicing the invention may be administered to deliver a dose of 1 ng/kg/day to 100 mg/kg/day. In one embodiment, the invention envisions administration of a dose that results in a concentration of the compound of the present invention between 1 μM and 100 μM in a mammal, preferably a human.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

Although the description of pharmaceutical compositions provided herein are principally directed to pharmaceutical compositions that are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as non-human primates, cattle, pigs, horses, sheep, cats, and dogs.

Typically, dosages which may be administered in a method of the invention to an animal, preferably a human, range in amount from 0.5 μg to about 50 mg per kilogram of body weight of the animal. While the precise dosage administered will vary depending upon any number of factors, including but not limited to, the type of animal and type of disease state being treated, the age of the animal and the route of administration, the dosage of the compound will preferably vary from about 1 μg to about 10 mg per kilogram of body weight of the animal. More preferably, the dosage will vary from about 3 μg to about 1 mg per kilogram of body weight of the animal.

Pharmaceutical compositions that are useful in the methods of the invention may be prepared, packaged, or sold in formulations suitable for oral, parenteral, topical, buccal, or another route of administration. Other contemplated formulations include projected nanoparticles, liposomal preparations, resealed erythrocytes containing the active ingredient, and immunologically-based formulations.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

In one embodiment, the compositions of the invention are formulated using one or more pharmaceutically acceptable excipients or carriers. In one embodiment, the pharmaceutical compositions of the invention comprise a therapeutically effective amount of a compound or conjugate of the invention and a pharmaceutically acceptable carrier. Pharmaceutically acceptable carriers that are useful, include, but are not limited to, glycerol, water, saline, ethanol and other pharmaceutically acceptable salt solutions such as phosphates and salts of organic acids. Examples of these and other pharmaceutically acceptable carriers are described in Remington's Pharmaceutical Sciences (1991, Mack Publication Co., New Jersey).

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it will be preferable to include isotonic agents, for example, sugars, sodium chloride, or polyalcohols such as mannitol and sorbitol, in the composition. Prolonged absorption of the injectable compositions may be brought about by including in the composition an agent that delays absorption, for example, aluminum monostearate or gelatin. In one embodiment, the pharmaceutically acceptable carrier is not DMSO alone.

Formulations may be employed in admixtures with conventional excipients, i.e., pharmaceutically acceptable organic or inorganic carrier substances suitable for oral, vaginal, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other analgesic agents.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” that may be included in the pharmaceutical compositions of the invention are known in the art and described, for example in Genaro, ed. (1985, Remington's Pharmaceutical Sciences, Mack Publishing Co., Easton, Pa.), which is incorporated herein by reference.

The composition of the invention may comprise a preservative from about 0.005% to 2.0% by total weight of the composition. The preservative is used to prevent spoilage in the case of exposure to contaminants in the environment. Examples of preservatives useful in accordance with the invention included but are not limited to those selected from the group consisting of benzyl alcohol, sorbic acid, parabens, imidurea and combinations thereof. A particularly preferred preservative is a combination of about 0.5% to 2.0% benzyl alcohol and 0.05% to 0.5% sorbic acid.

The composition preferably includes an anti-oxidant and a chelating agent that inhibits the degradation of the compound. Preferred antioxidants for some compounds are BHT, BHA, alpha-tocopherol and ascorbic acid in the preferred range of about 0.01% to 0.3% and more preferably BHT in the range of 0.03% to 0.1% by weight by total weight of the composition. Preferably, the chelating agent is present in an amount of from 0.01% to 0.5% by weight by total weight of the composition. Particularly preferred chelating agents include edetate salts (e.g. disodium edetate) and citric acid in the weight range of about 0.01% to 0.20% and more preferably in the range of 0.02% to 0.10% by weight by total weight of the composition. The chelating agent is useful for chelating metal ions in the composition that may be detrimental to the shelf life of the formulation. While BHT and disodium edetate are the particularly preferred antioxidant and chelating agent respectively for some compounds, other suitable and equivalent antioxidants and chelating agents may be substituted therefore as would be known to those skilled in the art.

Liquid suspensions may be prepared using conventional methods to achieve suspension of the active ingredient in an aqueous or oily vehicle. Aqueous vehicles include, for example, water, and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethylcellulose. Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g., polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin, and acacia. Known preservatives include, but are not limited to, methyl, ethyl, or n-propyl-para-hydroxybenzoates, ascorbic acid, and sorbic acid. Known sweetening agents include, for example, glycerol, propylene glycol, sorbitol, sucrose, and saccharin. Known thickening agents for oily suspensions include, for example, beeswax, hard paraffin, and cetyl alcohol.

Liquid solutions of the active ingredient in aqueous or oily solvents may be prepared in substantially the same manner as liquid suspensions, the primary difference being that the active ingredient is dissolved, rather than suspended in the solvent. As used herein, an “oily” liquid is one which comprises a carbon-containing liquid molecule and which exhibits a less polar character than water. Liquid solutions of the pharmaceutical composition of the invention may comprise each of the components described with regard to liquid suspensions, it being understood that suspending agents will not necessarily aid dissolution of the active ingredient in the solvent. Aqueous solvents include, for example, water, and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation of the invention may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, and a preservative. Additional excipients, such as fillers and sweetening, flavoring, or coloring agents, may also be included in these formulations.

A pharmaceutical composition of the invention may also be prepared, packaged, or sold in the form of oil-in-water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally-occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

Methods for impregnating or coating a material with a chemical composition are known in the art, and include, but are not limited to methods of depositing or binding a chemical composition onto a surface, methods of incorporating a chemical composition into the structure of a material during the synthesis of the material (i.e., such as with a physiologically degradable material), and methods of absorbing an aqueous or oily solution or suspension into an absorbent material, with or without subsequent drying.

Controlled- or sustained-release formulations of a composition of the invention may be made using conventional technology, in addition to the disclosure set forth elsewhere herein. In some cases, the dosage forms to be used can be provided as slow or controlled-release of one or more active ingredients therein using, for example, hydropropylmethyl cellulose, other polymer matrices, gels, permeable membranes, osmotic systems, multilayer coatings, microparticles, liposomes, or microspheres or a combination thereof to provide the desired release profile in varying proportions. Suitable controlled-release formulations known to those of ordinary skill in the art, including those described herein, can be readily selected for use with the compositions of the invention.

Controlled-release of an active ingredient can be stimulated by various inducers, for example pH, temperature, enzymes, water, or other physiological conditions or compounds. The term “controlled-release component” in the context of the present invention is defined herein as a compound or compounds, including, but not limited to, polymers, polymer matrices, gels, permeable membranes, liposomes, nanoparticles, or microspheres or a combination thereof that facilitates the controlled-release of the active ingredient.

### Administration/Dosing

The regimen of administration may affect what constitutes an effective amount. The therapeutic formulations may be administered to the subject either prior to or after a diagnosis of disease. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions of the present invention to a subject, preferably a mammal, more preferably a human, may be carried out using known procedures, at dosages and for periods of time effective to prevent or treat disease. An effective amount of the therapeutic compound necessary to achieve a therapeutic effect may vary according to factors such as the activity of the particular compound employed; the time of administration; the rate of excretion of the compound; the duration of the treatment; other drugs, compounds or materials used in combination with the compound; the state of the disease or disorder, age, sex, weight, condition, general health and prior medical history of the subject being treated, and like factors well-known in the medical arts. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound of the invention is from about 1 and 5,000 mg/kg of body weight/per day. One of ordinary skill in the art would be able to study the relevant factors and make the determination regarding the effective amount of the therapeutic compound without undue experimentation.

The compound may be administered to an animal as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the disease being treated, the type and age of the animal, etc. The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Actual dosage levels of the active ingredients in the pharmaceutical compositions of this invention may be varied so as to obtain an amount of the active ingredient that is effective to achieve the desired therapeutic response for a particular subject, composition, and mode of administration, without being toxic to the subject.

A medical doctor, e.g., physician or veterinarian, having ordinary skill in the art may readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the subjects to be treated; each unit containing a predetermined quantity of therapeutic compound calculated to produce the desired therapeutic effect in association with the required pharmaceutical vehicle. The dosage unit forms of the invention are dictated by and directly dependent on (a) the unique characteristics of the therapeutic compound and the particular therapeutic effect to be achieved, and (b) the limitations inherent in the art of compounding/formulating such a therapeutic compound for the treatment of a disease in a subject.

In one embodiment, the compositions of the invention are administered to the subject in dosages that range from one to five times per day or more. In another embodiment, the compositions of the invention are administered to the subject in range of dosages that include, but are not limited to, once every day, every two, days, every three days to once a week, and once every two weeks. It will be readily apparent to one skilled in the art that the frequency of administration of the various combination compositions of the invention will vary from subject to subject depending on many factors including, but not limited to, age, disease or disorder to be treated, gender, overall health, and other factors. Thus, the invention should not be construed to be limited to any particular dosage regime and the precise dosage and composition to be administered to any subject will be determined by the attending physical taking all other factors about the subject into account.

Compounds of the invention for administration may be in the range of from about 1 mg to about 10,000 mg, about 20 mg to about 9,500 mg, about 40 mg to about 9,000 mg, about 75 mg to about 8,500 mg, about 150 mg to about 7,500 mg, about 200 mg to about 7,000 mg, about 3050 mg to about 6,000 mg, about 500 mg to about 5,000 mg, about 750 mg to about 4,000 mg, about 1 mg to about 3,000 mg, about 10 mg to about 2,500 mg, about 20 mg to about 2,000 mg, about 25 mg to about 1,500 mg, about 50 mg to about 1,000 mg, about 75 mg to about 900 mg, about 100 mg to about 800 mg, about 250 mg to about 750 mg, about 300 mg to about 600 mg, about 400 mg to about 500 mg, and any and all whole or partial increments therebetween.

In some embodiments, the dose of a compound of the invention is from about 1 mg and about 2,500 mg. In some embodiments, a dose of a compound of the invention used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in some embodiments, a dose of a second compound (i.e., a drug used for treating the same or another disease as that treated by the compositions of the invention) as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In one embodiment, the present invention is directed to a packaged pharmaceutical composition comprising a container holding a therapeutically effective amount of a composition of the invention, alone or in combination with a second pharmaceutical agent; and instructions for using the composition to treat, prevent, or reduce one or more symptoms of a disease in a subject.

The term “container” includes any receptacle for holding the pharmaceutical composition. For example, in one embodiment, the container is the packaging that contains the pharmaceutical composition. In other embodiments, the container is not the packaging that contains the pharmaceutical composition, i.e., the container is a receptacle, such as a box or vial that contains the packaged pharmaceutical composition or unpackaged pharmaceutical composition and the instructions for use of the pharmaceutical composition. Moreover, packaging techniques are well known in the art. It should be understood that the instructions for use of the pharmaceutical composition may be contained on the packaging containing the pharmaceutical composition, and as such the instructions form an increased functional relationship to the packaged product. However, it should be understood that the instructions may contain information pertaining to the compound's ability to perform its intended function, e.g., treating or preventing a disease in a subject.

### Routes of Administration

Routes of administration of any of the compositions of the invention include oral, nasal, rectal, parenteral, sublingual, transdermal, transmucosal (e.g., sublingual, lingual, (trans)buccal, (trans)urethral, vaginal (e.g., trans- and perivaginally), (intra)nasal, and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present invention are not limited to the particular formulations and compositions that are described herein.

### Parenteral Administration

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, intraocular, intravitreal, subcutaneous, intraperitoneal, intramuscular, intrasternal injection, intratumoral, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e. powder or granular) form for reconstitution with a suitable vehicle (e.g. sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butanediol, for example. Other acceptable diluents and solvents include, but are not limited to, Ringer's solution, isotonic sodium chloride solution, and fixed oils such as synthetic mono- or di-glycerides. Other parentally-administrable formulations that are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

### Topical Administration

A pharmaceutical composition of the invention may be prepared, packaged, or sold in a formulation suitable for topical administration. There are several advantages to delivering compounds, including drugs or other therapeutic agents, into the skin (dermal drug delivery) or into the body through the skin (transdermal drug delivery). Transdermal compound delivery offers an attractive alternative to injections and oral medications. Dermal compound delivery offers an efficient way to deliver a compound to the skin of a mammal, and preferably a human, and provides a method of treatment of the skin, or otherwise provides a method of affecting the skin, without the need to break or damage the outer layer of the skin. In the present invention, dermal delivery, by way of a dermally-acting compound of the invention, provides these advantages for treatment of a skin-related condition, disorder or disease.

A number of compounds, including some drugs, will penetrate the skin effectively simply because the molecules are relatively small and potent at small doses of 0.1 mg to 15 mg/day (Kanikkannan et al., 2000, Curr. Med. Chem. 7:593-608). Many other compounds and drugs can be delivered only when an additional enhancement system is provided to “force” them to pass through the skin. Among several methods of transdermal drug delivery are electroporation, sonophoresis, iontophoresis, permeation enhancers (cyclodextrins), and liposomes. While the aforementioned methods are also included in the present invention for dermal delivery of the compounds of the invention, liposomes represent a preferred dermal delivery method.

The composition of the invention may consist of the active ingredient alone, in a form suitable for administration to a subject, or the composition may comprise at least one active ingredient and one or more pharmaceutically acceptable carriers, one or more additional ingredients, or some combination of these. The active ingredient may be present in the composition in the form of a physiologically acceptable ester or salt, such as in combination with a physiologically acceptable cation or anion, as is well known in the art. Compositions of the invention will also be understood to encompass pharmaceutical compositions useful for treatment of other conditions, disorders and diseases associated with the skin.

In one aspect, a dermal delivery vehicle of the invention is a composition comprising at least one first compound that can facilitate dermal delivery of at least one second compound associated with, or in close physical proximity to, the composition comprising the first compound. As will be understood by the skilled artisan, when armed with the disclosure set forth herein, such delivery vehicles include, but should not be limited to, liposomes, nanosomes, phospholipid-based non-liposome compositions (eg., selected cochleates), among others.

Formulations suitable for topical administration include, but are not limited to, liquid or semi-liquid preparations such as liniments, lotions, oil-in-water or water-in-oil emulsions such as creams, ointments or pastes, and solutions or suspensions. Topically-administrable formulations may, for example, comprise from about 0.001% to about 90% (w/w) active ingredient, although the concentration of the active ingredient may be as high as the solubility limit of the active ingredient in the solvent. Formulations for topical administration may further comprise one or more of the additional ingredients described herein.

In one aspect of the invention, a dermal delivery system includes a liposome delivery system, and that the present invention should not be construed to be limited to any particular liposome delivery system. Based on the disclosure set forth herein, the skilled artisan will understand how to identify a liposome delivery system as being useful in the present invention.

The present invention also encompasses the improvement of dermal and transdermal drug delivery through the use of penetration enhancers (also called sorption promoters or accelerants), which penetrate into skin to reversibly decrease the barrier resistance. Many compounds are known in the art for penetration enhancing activity, including sulphoxides (such as dimethylsulphoxide, DMSO), azones (e.g. laurocapram), pyrrolidones (for example 2-pyrrolidone, 2P), alcohols and alkanols (ethanol, or decanol), glycols (for example propylene glycol, PG, a common excipient in topically applied dosage forms), surfactants (also common in dosage forms) and terpenes. Other enhancers include oleic acid, oleyl alcohol, ethoxydiglycol, laurocapram, alkanecarboxylic acids, dimethylsulfoxide, polar lipids, or N-methyl-2-pyrrolidone.

In alternative embodiments, the topically active pharmaceutical or cosmetic composition may be optionally combined with other ingredients such as moisturizers, cosmetic adjuvants, anti-oxidants, chelating agents, surfactants, foaming agents, conditioners, humectants, wetting agents, emulsifying agents, fragrances, viscosifiers, buffering agents, preservatives, sunscreens and the like. In another embodiment, a permeation or penetration enhancer is included in the composition and is effective in improving the percutaneous penetration of the active ingredient into and through the stratum corneum with respect to a composition lacking the permeation enhancer. Various permeation enhancers, including oleic acid, oleyl alcohol, ethoxydiglycol, laurocapram, alkanecarboxylic acids, dimethylsulfoxide, polar lipids, or N-methyl-2-pyrrolidone, are known to those of skill in the art.

In another aspect, the composition may further comprise a hydrotropic agent, which functions to increase disorder in the structure of the stratum corneum, and thus allows increased transport across the stratum corneum. Various hydrotropic agents such as isopropyl alcohol, propylene glycol, or sodium xylene sulfonate, are known to those of skill in the art. The compositions of this invention may also contain active amounts of retinoids (i.e., compounds that bind to any members of the family of retinoid receptors), including, for example, tretinoin, retinol, esters of tretinoin and/or retinol and the like.

The composition of the invention may comprise a preservative from about 0.005% to 2.0% by total weight of the composition. The preservative is used to prevent spoilage in the case of an aqueous gel because of repeated patient use when it is exposed to contaminants in the environment from, for example, exposure to air or the patient's skin, including contact with the fingers used for applying a composition of the invention such as a therapeutic gel or cream. Examples of preservatives useful in accordance with the invention included but are not limited to those selected from the group consisting of benzyl alcohol, sorbic acid, parabens, imidurea and combinations thereof. A particularly preferred preservative is a combination of about 0.5% to 2.0% benzyl alcohol and 0.05% to 0.5% sorbic acid.

The composition preferably includes an antioxidant and a chelating agent which inhibit the degradation of the compound for use in the invention in the aqueous gel formulation. Preferred antioxidants for some compounds are BHT, BHA, alpha-tocopherol and ascorbic acid in the preferred range of about 0.01% to 5% and BHT in the range of 0.01% to 1% by weight by total weight of the composition. Preferably, the chelating agent is present in an amount of from 0.01% to 0.5% by weight by total weight of the composition. Particularly preferred chelating agents include edetate salts (e.g. disodium edetate) and citric acid in the weight range of about 0.01% to 0.20% and more preferably in the range of 0.02% to 0.10% by weight by total weight of the composition. The chelating agent is useful for chelating metal ions in the composition which may be detrimental to the shelf life of the formulation. While BHT and disodium edetate are the particularly preferred antioxidant and chelating agent respectively for some compounds, other suitable and equivalent antioxidants and chelating agents may be substituted therefore as would be known to those skilled in the art.

Additional components may include, but should not be limited to those including water, oil (eg., olive oil/PEG7), biovera oil, wax (eg., jojoba wax), squalene, myristate (eg., isopropyl myristate), triglycerides (eg., caprylic triglyceride), Solulan 98, cocoa butter, shea butter, alcohol (eg., behenyl alcohol), stearate (eg., glycerol-monostearate), chelating agents (eg., EDTA), propylene glycol, SEPIGEL (Seppic, Inc., Fairfield, N.J.), silicone and silicone derivatives (eg., dimethicone, cyclomethicone), vitamins (eg., vitamin E), among others.

### Oral Administration

For oral application, particularly suitable are tablets, dragees, liquids, drops, suppositories, or capsules, caplets and gelcaps. Other formulations suitable for oral administration include, but are not limited to, a powdered or granular formulation, an aqueous or oily suspension, an aqueous or oily solution, a paste, a gel, toothpaste, a mouthwash, a coating, an oral rinse, or an emulsion. The compositions intended for oral use may be prepared according to any method known in the art and such compositions may contain one or more agents selected from the group consisting of inert, non-toxic pharmaceutically excipients that are suitable for the manufacture of tablets. Such excipients include, for example an inert diluent such as lactose; granulating and disintegrating agents such as cornstarch; binding agents such as starch; and lubricating agents such as magnesium stearate.

Tablets may be non-coated or they may be coated using known methods to achieve delayed disintegration in the gastrointestinal tract of a subject, thereby providing sustained release and absorption of the active ingredient. By way of example, a material such as glyceryl monostearate or glyceryl distearate may be used to coat tablets. Further by way of example, tablets may be coated using methods described in U.S. Pat. Nos. 4,256,108; 4,160,452; and 4,265,874 to form osmotically controlled release tablets. Tablets may further comprise a sweetening agent, a flavoring agent, a coloring agent, a preservative, or some combination of these in order to provide for pharmaceutically elegant and palatable preparation.

Hard capsules comprising the active ingredient may be made using a physiologically degradable composition, such as gelatin. Such hard capsules comprise the active ingredient, and may further comprise additional ingredients including, for example, an inert solid diluent such as calcium carbonate, calcium phosphate, or kaolin.

Soft gelatin capsules comprising the active ingredient may be made using a physiologically degradable composition, such as gelatin. Such soft capsules comprise the active ingredient, which may be mixed with water or an oil medium such as peanut oil, liquid paraffin, or olive oil.

For oral administration, the compositions of the invention may be in the form of tablets or capsules prepared by conventional means with pharmaceutically acceptable excipients such as binding agents; fillers; lubricants; disintegrates; or wetting agents. If desired, the tablets may be coated using suitable methods and coating materials such as OPADRY™ film coating systems available from Colorcon, West Point, Pa. (e.g., OPADRY™ OY Type, OYC Type, Organic Enteric OY-P Type, Aqueous Enteric OY-A Type, OY-PM Type and OPADRY™ White, 32K18400).

Liquid preparation for oral administration may be in the form of solutions, syrups or suspensions. The liquid preparations may be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl para-hydroxy benzoates or sorbic acid). Liquid formulations of a pharmaceutical composition of the invention which are suitable for oral administration may be prepared, packaged, and sold either in liquid form or in the form of a dry product intended for reconstitution with water or another suitable vehicle prior to use.

A tablet comprising the active ingredient may, for example, be made by compressing or molding the active ingredient, optionally with one or more additional ingredients. Compressed tablets may be prepared by compressing, in a suitable device, the active ingredient in a free-flowing form such as a powder or granular preparation, optionally mixed with one or more of a binder, a lubricant, an excipient, a surface active agent, and a dispersing agent. Molded tablets may be made by molding, in a suitable device, a mixture of the active ingredient, a pharmaceutically acceptable carrier, and at least sufficient liquid to moisten the mixture. Pharmaceutically acceptable excipients used in the manufacture of tablets include, but are not limited to, inert diluents, granulating and disintegrating agents, binding agents, and lubricating agents. Known dispersing agents include, but are not limited to, potato starch and sodium starch glycollate. Known surface-active agents include, but are not limited to, sodium lauryl sulphate. Known diluents include, but are not limited to, calcium carbonate, sodium carbonate, lactose, microcrystalline cellulose, calcium phosphate, calcium hydrogen phosphate, and sodium phosphate. Known granulating and disintegrating agents include, but are not limited to, corn starch and alginic acid. Known binding agents include, but are not limited to, gelatin, acacia, pre-gelatinized maize starch, polyvinylpyrrolidone, and hydroxypropyl methylcellulose. Known lubricating agents include, but are not limited to, magnesium stearate, stearic acid, silica, and talc.

Granulating techniques are well known in the pharmaceutical art for modifying starting powders or other particulate materials of an active ingredient. The powders are typically mixed with a binder material into larger permanent free-flowing agglomerates or granules referred to as a “granulation.” For example, solvent-using “wet” granulation processes are generally characterized in that the powders are combined with a binder material and moistened with water or an organic solvent under conditions resulting in the formation of a wet granulated mass from which the solvent must then be evaporated.

Melt granulation generally consists in the use of materials that are solid or semi-solid at room temperature (i.e. having a relatively low softening or melting point range) to promote granulation of powdered or other materials, essentially in the absence of added water or other liquid solvents. The low melting solids, when heated to a temperature in the melting point range, liquefy to act as a binder or granulating medium. The liquefied solid spreads itself over the surface of powdered materials with which it is contacted, and on cooling, forms a solid granulated mass in which the initial materials are bound together. The resulting melt granulation may then be provided to a tablet press or be encapsulated for preparing the oral dosage form. Melt granulation improves the dissolution rate and bioavailability of an active (i.e. drug) by forming a solid dispersion or solid solution.

U.S. Pat. No. 5,169,645 discloses directly compressible wax-containing granules having improved flow properties. The granules are obtained when waxes are admixed in the melt with certain flow improving additives, followed by cooling and granulation of the admixture. In certain embodiments, only the wax itself melts in the melt combination of the wax(es) and additives(s), and in other cases both the wax(es) and the additives(s) will melt.

The present invention also includes a multi-layer tablet comprising a layer providing for the delayed release of one or more compounds of the invention, and a further layer providing for the immediate release of a medication for treatment of a disease. Using a wax/pH-sensitive polymer mix, a gastric insoluble composition may be obtained in which the active ingredient is entrapped, ensuring its delayed release.

### Buccal Administration

A pharmaceutical composition of the invention may be prepared, packaged, or sold in a formulation suitable for buccal administration. Such formulations may, for example, be in the form of tablets or lozenges made using conventional methods, and may, for example, 0.1 to 20% (w/w) active ingredient, the balance comprising an orally dissolvable or degradable composition and, optionally, one or more of the additional ingredients described herein. Alternately, formulations suitable for buccal administration may comprise a powder or an aerosolized or atomized solution or suspension comprising the active ingredient. Such powdered, aerosolized, or aerosolized formulations, when dispersed, preferably have an average particle or droplet size in the range from about 0.1 to about 200 nanometers, and may further comprise one or more of the additional ingredients described herein.

### Rectal Administration

A pharmaceutical composition of the invention may be prepared, packaged, or sold in a formulation suitable for rectal administration. Such a composition may be in the form of, for example, a suppository, a retention enema preparation, and a solution for rectal or colonic irrigation.

Suppository formulations may be made by combining the active ingredient with a non-irritating pharmaceutically acceptable excipient which is solid at ordinary room temperature (i.e., about 20° C.) and which is liquid at the rectal temperature of the subject (i.e., about 37° C. in a healthy human). Suitable pharmaceutically acceptable excipients include, but are not limited to, cocoa butter, polyethylene glycols, and various glycerides. Suppository formulations may further comprise various additional ingredients including, but not limited to, antioxidants, and preservatives.

Retention enema preparations or solutions for rectal or colonic irrigation may be made by combining the active ingredient with a pharmaceutically acceptable liquid carrier. As is well known in the art, enema preparations may be administered using, and may be packaged within, a delivery device adapted to the rectal anatomy of the subject. Enema preparations may further comprise various additional ingredients including, but not limited to, antioxidants, and preservatives.

### Vaginal Administration

A pharmaceutical composition of the invention may be prepared, packaged, or sold in a formulation suitable for vaginal administration. With respect to the vaginal or perivaginal administration of the compounds of the invention, dosage forms may include vaginal suppositories, creams, ointments, liquid formulations, pessaries, tampons, gels, pastes, foams or sprays. The suppository, solution, cream, ointment, liquid formulation, pessary, tampon, gel, paste, foam or spray for vaginal or perivaginal delivery comprises a therapeutically effective amount of the selected active agent and one or more conventional nontoxic carriers suitable for vaginal or perivaginal drug administration. The vaginal or perivaginal forms of the present invention may be manufactured using conventional processes as disclosed in Remington: The Science and Practice of Pharmacy, supra (see also drug formulations as adapted in U.S. Pat. Nos. 6,515,198; 6,500,822; 6,417,186; 6,416,779; 6,376,500; 6,355,641; 6,258,819; 6,172,062; and 6,086,909). The vaginal or perivaginal dosage unit may be fabricated to disintegrate rapidly or over a period of several hours. The time period for complete disintegration may be in the range of from about 10 minutes to about 6 hours, e.g., less than about 3 hours.

Methods for impregnating or coating a material with a chemical composition are known in the art, and include, but are not limited to methods of depositing or binding a chemical composition onto a surface, methods of incorporating a chemical composition into the structure of a material during the synthesis of the material (i.e., such as with a physiologically degradable material), and methods of absorbing an aqueous or oily solution or suspension into an absorbent material, with or without subsequent drying.

Douche preparations or solutions for vaginal irrigation may be made by combining the active ingredient with a pharmaceutically acceptable liquid carrier. As is well known in the art, douche preparations may be administered using, and may be packaged within, a delivery device adapted to the vaginal anatomy of the subject.

Douche preparations may further comprise various additional ingredients including, but not limited to, antioxidants, antibiotics, antifungal agents, and preservatives.

### Additional Administration Forms

Additional dosage forms of this invention include dosage forms as described in U.S. Pat. Nos. 6,340,475; 6,488,962; 6,451,808; 5,972,389; 5,582,837 and 5,007,790. Additional dosage forms of this invention also include dosage forms as described in U.S. Patent Applications Nos. 20030147952, 20030104062, 20030104053, 20030044466, 20030039688, and 20020051820. Additional dosage forms of this invention also include dosage forms as described in PCT Applications Nos. WO 03/35041, WO 03/35040, WO 03/35029, WO 03/35177, WO 03/35039, WO 02/96404, WO 02/32416, WO 01/97783, WO 01/56544, WO 01/32217, WO 98/55107, WO 98/11879, WO 97/47285, WO 93/18755, and WO 90/11757.

### Kits of the Invention

The invention also includes a kit comprising a LYN kinase inhibitor and an instructional material that describes, for instance, administering the LYN kinase inhibitor to a subject as a prophylactic or therapeutic treatment or a non-treatment use as described elsewhere herein. In an embodiment, the kit further comprises a (preferably sterile) pharmaceutically acceptable carrier suitable for dissolving or suspending the therapeutic composition, comprising a LYN kinase inhibitor, for instance, prior to administering the molecule to a subject. Optionally, the kit comprises an applicator for administering the inhibitor.

In another embodiment, the kit comprises compositions required for the detection of LYN kinase expression, function, or activity in a sample invention and an instructional material that describes, for instance, the method for detecting LYN kinase expression, function, or activity in a biological sample as described elsewhere herein.

Those skilled in the art will recognize, or be able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that wherever values and ranges are provided herein, all values and ranges encompassed by these values and ranges, are meant to be encompassed within the scope of the present invention. Moreover, all values that fall within these ranges, as well as the upper or lower limits of a range of values, are also contemplated by the present application.

The following examples further illustrate aspects of the present invention. However, they are in no way a limitation of the teachings or disclosure of the present invention as set forth herein.

### EXAMPLES

The invention is now described with reference to the following Examples. These Examples are provided for the purpose of illustration only, and the invention is not limited to these Examples, but rather encompasses all variations that are evident as a result of the teachings provided herein.

The materials and methods employed in the experiments and the results of the experiments presented in this Example are now described.

### Example 1

### Testing Procedure

In order to identify novel LYN kinase inhibitors using the ChemVassa methodology, currently known kinase inhibitors were analyzed using ChemVassa. In this study, EGFR inhibitors were used, and dasatinib (N-(2-chloro-6-methylphenyl)-2-[[6-[4-(2-hydroxyethyl)-1-piperazinyl]-2-methyl-4-pyrimidinyl]amino]-5-thiazole carboxamide) was the template compound.

The template compound was used as a query within the ChemVassa Search Engine. The search was run through the 16 million compound database over the period of 14 hours. This study allowed the identification of a set of initial hits within the ChemVassa Search Engine, several of which had CV similarity scores less than initial cut-off of 8. The hits were then restricted to those compounds with good “rule of five” (RO5) characteristics.

The identified hits were further refined to select those compounds that had molecular weights that would allow medicinal chemistry manipulation, and were also readily available from commercial vendors. This effort resulted in the identification of ten compounds, which were then submitted to in silico docking and modeling. All ten compounds displayed reasonable binding characteristics, and three of those were selected for further bench testing based on their activities and availabilities.

### Example 2

### Validation Procedure

The three compounds identified in Example 1 were tested for kinase activity at Millipore (Dundee UK). The compounds were shipped as 10 mM stocks in DMSO as per Millipore testing standards. The testing consisted of two initial phases:

(a) Profiling against a set of approximately 60 kinases, selected as representative of all kinases and current kinase drug targets, to determine the selectivity of the compounds; and,
(b) IC₅₀ testing againstr EGFR, the kinase target of the initial search template molecule. Initial testing showed that no compound in the set inhibited EGFR.

Initial testing also showed that two of the compounds inhibited other kinases in a competitive binding assay. In particular, compound VI301 showed good inhibition of LYN kinase at 100 μM concentration (Table 1).

TABLE 1

| | Inhibition by VI301 |
|---|---|
| | (residual activity at 100 μM |
| Kinase | inhibitor) |
| Lyn(h) | 5% |
| Pim-1(h) | 88% |
| WNK3(h) | 97% |

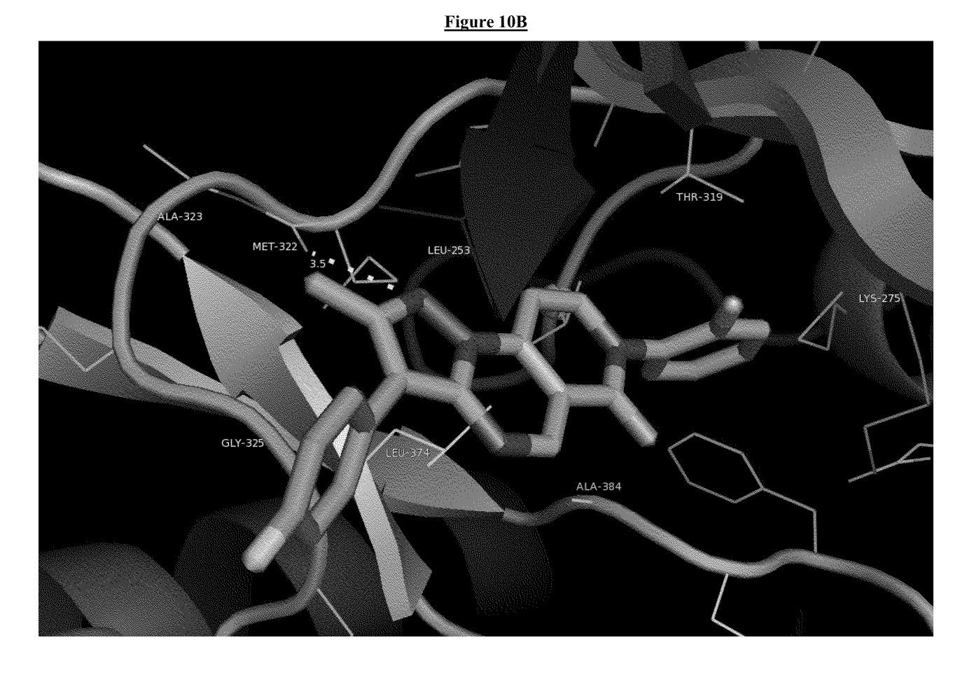

VI301 (FIG. 10; also known as 2-methyl-3,7-diphenylpyrazolo[1,5-a]pyrido[3,4-e]pyrimidin-6(7H)-one), has the following characteristics: xLogP = 4.45; H-bond Donors = 0; H-bond acceptors = 5; Charge = 0; MW = 388.377; Number rotatable bonds = 2.

VI301 was submitted for IC₅₀ determinations, and docked and modeled in LYN kinase. Docking of VI301 confirmed the exceptional affinity of this compound for LYN kinase. Tanimoto coefficients for VI301 versus dasatinib were about 20.29%, suggesting that the commercially available programs other than ChemVassa would not have identified this compound as a potential LYN kinase inhibitor.

The experiments described herein indicate that the ChemVassa protocol allows the identification of novel and potent kinase inhibitors. ChemVassa may be used to profile existing drugs, wherein such profiles are further investigated within the ChemVassa Search Engine and new candidates for bench testing are identified. ChemVassa is an abstraction of a molecule, and captures relevant information about the molecule that other current abstraction and search tools do not. Thus, ChemVassa allows for the extension of the range of potential hits generated for bench testing. The success ratio with ChemVassa validates the best case scenarios for the functionality of ChemVassa as a drug discovery tool. Lead molecules identified by ChemVassa would not have been found using current hit and hit-to-lead protocols and procedures. This characteristic allows for generating new leads and analyzing existing target pools for novelty.

The disclosures of each and every patent, patent application, and publication cited herein are hereby incorporated herein by reference in their entirety.

While the invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A compound of formula (I): 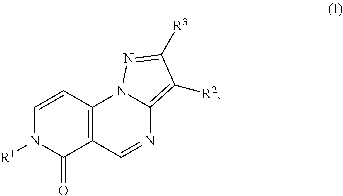
or a salt thereof, wherein:
R¹ and R⁶ are independently unsubstituted or substituted alkyl, unsubstituted or substituted aryl, or unsubstituted or substituted heteroaryl; and
R³ is H or C₁-C₆ alkyl;
with the proviso that the compound of formula (I) is not 7-(2-fluorophenyl)-3-(4-fluorophenyl)-2-methylpyrazolo[1,5-a]pyrido[3,4-e]pyrimidin-6(7H)-one; 3-(4-chlorophenyl)-7-(2-fluorophenyl)-2-methylpyrazolo[1,5-a]pyrido[3,4-e]pyrimidin-6(7H)-one; 7-(2-fluorophenyl)-2-methyl-3-phenylpyrazolo[1,5-a]pyrido[3,4-e]pyrimidin-6(7H)-one; 7-(3-fluorophenyl)-2-methyl-3-phenylpyrazolo[1,5-a]pyrido[3,4-e]pyrimidin-6(7H)-one; 7-(3-chloro-4-fluorophenyl)-2-methyl-3-phenylpyrazolo[1,5-a]pyrido[3,4-e]pyrimidin-6(7H)-one; 7-(3,4-difluorophenyl)-2-methyl-3-phenylpyrazolo[1,5-a]pyrido[3,4-e]pyrimidin-6(7H)-one; 7-(3-chlorophenyl)-3-(4-fluorophenyl)-2-methylpyrazolo[1,5-a]pyrido[3,4-e]pyrimidin-6(7H)-one; 7-(2-chlorophenyl)-3-(4-fluorophenyl)-2-methylpyrazolo[1,5-a]pyrido[3,4-e]pyrimidin-6(7H)-one; 3-(4-fluorophenyl)-7-(2-methoxyphenyl)-2-methylpyrazolo[1,5-a]pyrido[3,4-e]pyrimidin-6(7H)-one; 2-(3-(4-fluorophenyl)-2-methyl-6-oxopyrazolo[1,5-a]pyrido[3,4-e]pyrimidin-7(6H)-yl)benzonitrile; 2-ethyl-3-(4-fluorophenyl)-7-phenylpyrazolo[1,5-a]pyrido[3,4-e]pyrimidin-6(7H)-one; or a salt thereof.

2. The compound of claim 1, wherein R¹ and R² are independently unsubstituted or substituted aryl, or unsubstituted or substituted heteroaryl.

3. The compound of claim 1, wherein R³ is H, methyl or ethyl.

4. A method of inhibiting LYN kinase activity in a subject in need thereof, the method comprising administering to the subject a pharmaceutical composition comprising a pharmaceutically acceptable carrier and a therapeutically effective amount of a compound of formula (II): 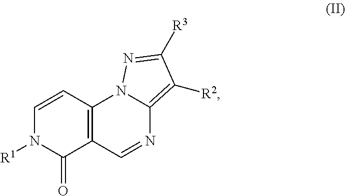
or a salt thereof, wherein:
R¹ and R⁶ are independently unsubstituted or substituted alkyl, unsubstituted or substituted aryl, or unsubstituted or substituted heteroaryl; and
R³ is H or C₁-C₆ alkyl;
whereby the administration of the composition inhibits LYN kinase activity in the subject.

5. The method of claim 4, wherein the compound of formula (II) is selected from the group consisting of:
7-(2-fluorophenyl)-3-(4-fluorophenyl)-2-methylpyrazolo[1,5-a]pyrido[3,4-e]pyrimidin-6(7H)-one: 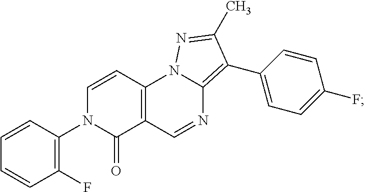 [SMILES: Cc1nn2c(ncc3c(=O)n(-c4ccccc4F)ccc32)c1-c1ccc(F)cc1]
3-(4-chlorophenyl)-7-(2-fluorophenyl)-2-methylpyrazolo[1,5-a]pyrido[3,4-e]pyrimidin-6(7H)-one: 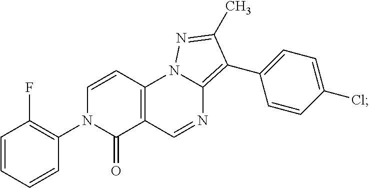 [SMILES: Cc1nn2c(ncc3c(=O)n(-c4ccccc4F)ccc32)c1-c1ccc(Cl)cc1]
7-(2-fluorophenyl)-2-methyl-3-phenylpyrazolo[1,5-a]pyrido[3,4-e]pyrimidin-6(7H)-one: 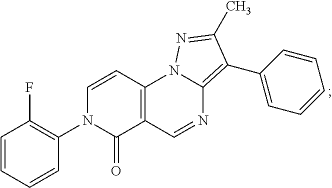 [SMILES: Cc1nn2c(ncc3c(=O)n(-c4ccccc4F)ccc32)c1-c1ccccc1]
7-(3-fluorophenyl)-2-methyl-3-phenylpyrazolo[1,5-a]pyrido[3,4-e]pyrimidin-6(7H)-one: 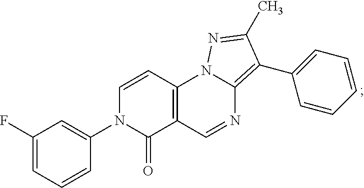 [SMILES: Cc1nn2c(ncc3c(=O)n(-c4cccc(F)c4)ccc32)c1-c1ccccc1]
7-(3-chloro-4-fluorophenyl)-2-methyl-3-phenylpyrazolo[1,5-a]pyrido[3,4-e]pyrimidin-6(7H)-one: 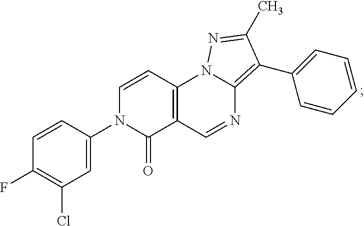 [SMILES: Cc1nn2c(ncc3c(=O)n(-c4ccc(F)c(Cl)c4)ccc32)c1-c1ccccc1]
7-(3,4-difluorophenyl)-2-methyl-3-phenylpyrazolo[1,5-a]pyrido[3,4-e]pyrimidin-6(7H)-one: 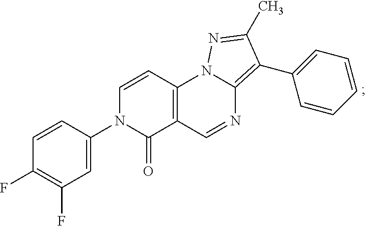 [SMILES: Cc1nn2c(ncc3c(=O)n(-c4ccc(F)c(F)c4)ccc32)c1-c1ccccc1]
7-(3-chlorophenyl)-3-(4-fluorophenyl)-2-methylpyrazolo[1,5-a]pyrido[3,4-e]pyrimidin-6(7H)-one: 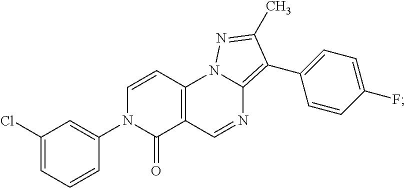 [SMILES: Cc1nn2c(ncc3c(=O)n(-c4cccc(Cl)c4)ccc32)c1-c1ccc(F)cc1]
7-(2-chlorophenyl)-3-(4-fluorophenyl)-2-methylpyrazolo[1,5-a]pyrido[3,4-e]pyrimidin-6(7H)-one: 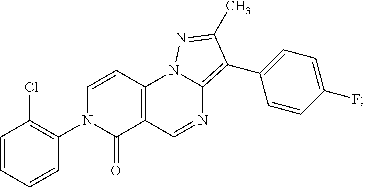 [SMILES: Cc1nn2c(ncc3c(=O)n(-c4ccccc4Cl)ccc32)c1-c1ccc(F)cc1]
3-(4-fluorophenyl)-7-(2-methoxyphenyl)-2-methylpyrazolo[1,5-a]pyrido[3,4-e]pyrimidin-6(7H)-one: 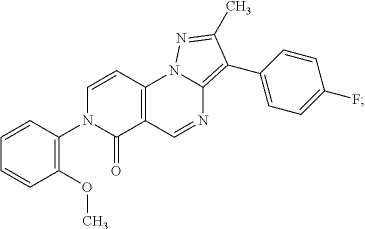 [SMILES: COc1ccccc1-n1ccc2c(cnc3c(-c4ccc(F)cc4)c(C)nn32)c1=O]
2-(3-(4-fluorophenyl)-2-methyl-6-oxopyrazolo[1,5-a]pyrido[3,4-e]pyrimidin-7(6H)-yl)benzonitrile: 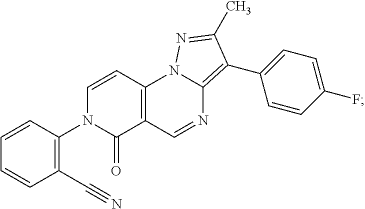 [SMILES: Cc1nn2c(ncc3c(=O)n(-c4ccccc4C#N)ccc32)c1-c1ccc(F)cc1]
2-ethyl-3-(4-fluorophenyl)-7-phenylpyrazolo[1,5-a]pyrido[3,4-e]pyrimidin-6(7H)-one: 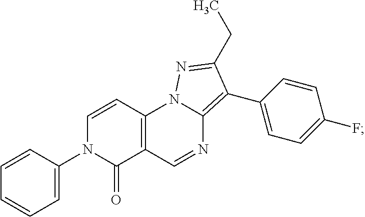 [SMILES: CCc1nn2c(ncc3c(=O)n(-c4ccccc4)ccc32)c1-c1ccc(F)cc1]
and a salt thereof.

6. The method of claim 4, wherein the subject is human.

7. A method of treating, ameliorating or preventing cancer in a subject in need thereof, the method comprising administering to the subject a pharmaceutical composition comprising a pharmaceutically acceptable carrier and a therapeutically effective amount of a compound of formula (II): 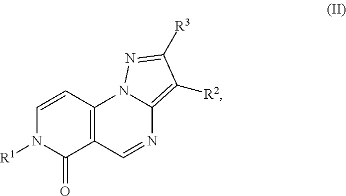
or a salt thereof, wherein:
R¹ and R⁶ are independently unsubstituted or substituted alkyl, unsubstituted or substituted aryl, or unsubstituted or substituted heteroaryl; and
R³ is H or C₁-C₆ alkyl;
wherein the cancer is dependent on LYN kinase activity, whereby administration of the composition inhibits the LYN kinase activity and treats, ameliorates or prevent the cancer in the subject.

8. The method of claim 7, wherein the compound of formula (II) is selected from the group consisting of:
7-(2-fluorophenyl)-3-(4-fluorophenyl)-2-methylpyrazolo[1,5-a]pyrido[3,4-e]pyrimidin-6(7H)-one: 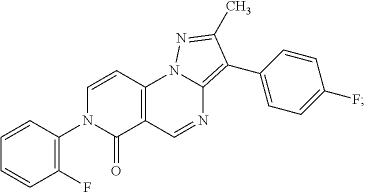 [SMILES: Cc1nn2c(ncc3c(=O)n(-c4ccccc4F)ccc32)c1-c1ccc(F)cc1]
3-(4-chlorophenyl)-7-(2-fluorophenyl)-2-methylpyrazolo[1,5-a]pyrido[3,4-e]pyrimidin-6(7H)-one: 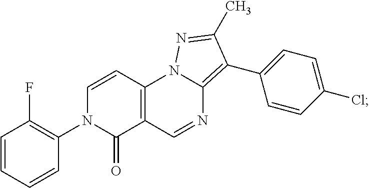 [SMILES: Cc1nn2c(ncc3c(=O)n(-c4ccccc4F)ccc32)c1-c1ccc(Cl)cc1]
7-(2-fluorophenyl)-2-methyl-3-phenylpyrazolo[1,5-a]pyrido[3,4-e]pyrimidin-6(7H)-one: 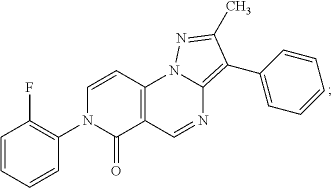 [SMILES: Cc1nn2c(ncc3c(=O)n(-c4ccccc4F)ccc32)c1-c1ccccc1]
7-(3-fluorophenyl)-2-methyl-3-phenylpyrazolo[1,5-a]pyrido[3,4-e]pyrimidin-6(7H)-one: 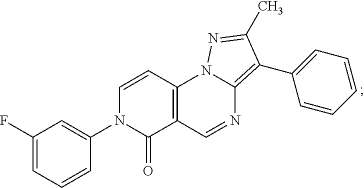 [SMILES: Cc1nn2c(ncc3c(=O)n(-c4cccc(F)c4)ccc32)c1-c1ccccc1]
7-(3-chloro-4-fluorophenyl)-2-methyl-3-phenylpyrazolo[1,5-a]pyrido[3,4-e]pyrimidin-6(7H)-one: 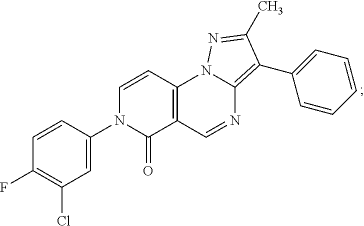 [SMILES: Cc1nn2c(ncc3c(=O)n(-c4ccc(F)c(Cl)c4)ccc32)c1-c1ccccc1]
7-(3,4-difluorophenyl)-2-methyl-3-phenylpyrazolo[1,5-a]pyrido[3,4-e]pyrimidin-6(7H)-one: 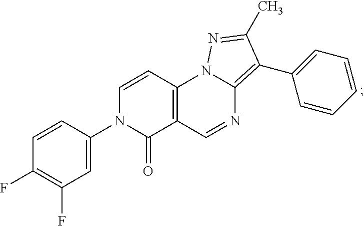 [SMILES: Cc1nn2c(ncc3c(=O)n(-c4ccc(F)c(F)c4)ccc32)c1-c1ccccc1]
7-(3-chlorophenyl)-3-(4-fluorophenyl)-2-methylpyrazolo[1,5-a]pyrido[3,4-e]pyrimidin-6(7H)-one: 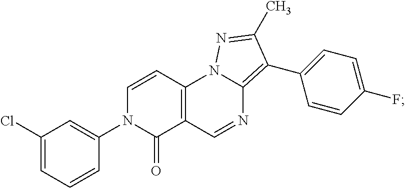 [SMILES: Cc1nn2c(ncc3c(=O)n(-c4cccc(Cl)c4)ccc32)c1-c1ccc(F)cc1]
7-(2-chlorophenyl)-3-(4-fluorophenyl)-2-methylpyrazolo[1,5-a]pyrido[3,4-e]pyrimidin-6(7H)-one: 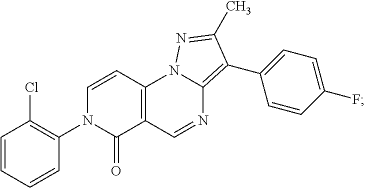 [SMILES: Cc1nn2c(ncc3c(=O)n(-c4ccccc4Cl)ccc32)c1-c1ccc(F)cc1]
3-(4-fluorophenyl)-7-(2-methoxyphenyl)-2-methylpyrazolo[1,5-a]pyrido[3,4-e]pyrimidin-6(7H)-one: 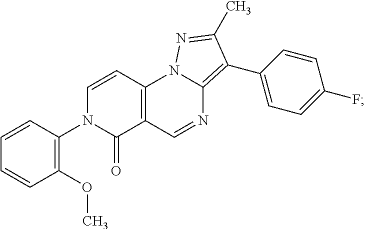 [SMILES: COc1ccccc1-n1ccc2c(cnc3c(-c4ccc(F)cc4)c(C)nn32)c1=O]
2-(3-(4-fluorophenyl)-2-methyl-6-oxopyrazolo[1,5-a]pyrido[3,4-e]pyrimidin-7(6H)-yl)benzonitrile: 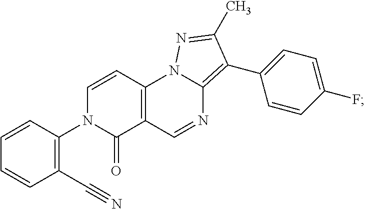 [SMILES: Cc1nn2c(ncc3c(=O)n(-c4ccccc4C#N)ccc32)c1-c1ccc(F)cc1]
2-ethyl-3-(4-fluorophenyl)-7-phenylpyrazolo[1,5-a]pyrido[3,4-e]pyrimidin-6(7H)-one: 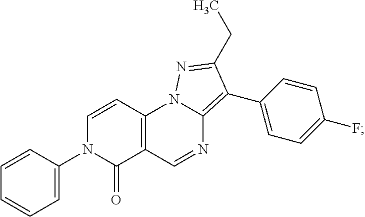 [SMILES: CCc1nn2c(ncc3c(=O)n(-c4ccccc4)ccc32)c1-c1ccc(F)cc1]
and a salt thereof.

9. The method of claim 7, wherein the subject is human.

10. The method of claim 7, wherein the cancer is selected from the group consisting of CML, B-CLL, glioblastoma, breast cancer, Ewing's sarcoma and prostate cancer.

11. The method of claim 10, wherein the CML cancer is resistant to 4-[(4-methylpiperazin-1-yl)methyl]-N-[4-methyl-3-[(4-pyridin-3-ylpyrimidin-2-yl)amino]phenyl]benzamide (imatinib).
